SEQUENCE LISTING (28 sequences)

SEQ ID NO: 1 - DNA, 23 bp, Homo sapiens
  source 1..23: mol_type = genomic DNA
tttgattgacatactttggaggc

SEQ ID NO: 2 - DNA, 23 bp, Homo sapiens
  source 1..23: mol_type = genomic DNA
cttactccaagatctatatcttg

SEQ ID NO: 3 - DNA, 23 bp, Homo sapiens
  source 1..23: mol_type = genomic DNA
atactttggaggcaagatataga

SEQ ID NO: 4 - DNA, 23 bp, Homo sapiens
  source 1..23: mol_type = genomic DNA
aggcaagatatagatcttggagt

SEQ ID NO: 5 - DNA, 23 bp, Homo sapiens
  source 1..23: mol_type = genomic DNA
gatatagatcttggagtaagtcg

SEQ ID NO: 6 - DNA, 23 bp, Homo sapiens
  source 1..23: mol_type = genomic DNA
tgactgaagtcaaatacttctcg

SEQ ID NO: 7 - DNA, 23 bp, Homo sapiens
  source 1..23: mol_type = genomic DNA
ttcatctagttgtaactgagcga

SEQ ID NO: 8 - DNA, 23 bp, Homo sapiens
  source 1..23: mol_type = genomic DNA
attcacctgtctcttcatctagt

SEQ ID NO: 9 - DNA, 23 bp, Homo sapiens
  source 1..23: mol_type = genomic DNA
ctcagttacaactagatgaagag

SEQ ID NO: 10 - DNA, 23 bp, Homo sapiens
  source 1..23: mol_type = genomic DNA
tgaattgggagaaattcacctgt

SEQ ID NO: 11 - DNA, 23 bp, Homo sapiens
  source 1..23: mol_type = genomic DNA
caactagatgaagagacaggtga

SEQ ID NO: 12 - DNA, 23 bp, Homo sapiens
  source 1..23: mol_type = genomic DNA
ataatagctcctcccaaacttgc

SEQ ID NO: 13 - DNA, 23 bp, Homo sapiens
  source 1..23: mol_type = genomic DNA
tttttcgctcagttacaactaga

SEQ ID NO: 14 - DNA, 23 bp, Homo sapiens
  source 1..23: mol_type = genomic DNA
caactagatgaagagacaggtga

SEQ ID NO: 15 - DNA, 2446 bp, Homo sapiens
  source 1..2446: mol_type = genomic DNA
gattaccgagtgccggggagcccggaggagccgccgacgcagccgccaccgccgccgccgccgccaccagagccgccctgtccgcgccgcgcctcggcagccggaacagggccgccgtcggggagccccaacacacggtccacagctcatcatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaa

SEQ ID NO: 16 - DNA, 9905 bp, Homo sapiens
  source 1..9905: mol_type = genomic DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 17 - protein, 1210 aa, Homo sapiens [sequence omitted]

SEQ ID NO: 18 - RNA, 95 nt, synthetic construct
  source 1..95: mol_type = other RNA
cttactccaagatctatatcgcaccgactcggtgccactttttcaagttgataacggactagcctattttaacttgctatttctagctctaaaac

SEQ ID NO: 19 - DNA, 20 bp, synthetic construct
  source 1..20: mol_type = other DNA
catggccttccgtgttccta

SEQ ID NO: 20 - DNA, 20 bp, synthetic construct
  source 1..20: mol_type = other DNA
acttggcaggtttctccagg

SEQ ID NO: 21 - DNA, 23 bp, synthetic construct
  source 1..23: mol_type = other DNA
tctcctctgacttcaacagcgac

SEQ ID NO: 22 - DNA, 22 bp, synthetic construct
  source 1..22: mol_type = other DNA
ccctgttgctgtagccaaattc

SEQ ID NO: 23 - DNA, 20 bp, synthetic construct
  source 1..20: mol_type = other DNA
tgatgtaccggttcgaggag

SEQ ID NO: 24 - DNA, 20 bp, synthetic construct
  source 1..20: mol_type = other DNA
ttgtcgatcagggtgctctt

SEQ ID NO: 25 - DNA, 20 bp, synthetic construct
  source 1..20: mol_type = other DNA
gatatagatcttggagtaag

SEQ ID NO: 26 - DNA, 20 bp, synthetic construct
  source 1..20: mol_type = other DNA
gcactaccagagctaactca

SEQ ID NO: 27 - DNA, 26 bp, synthetic construct
  source 1..26: mol_type = other DNA
caccatcaacagtggcataatgtgaa

SEQ ID NO: 28 - DNA, 27 bp, synthetic construct
  source 1..27: mol_type = other DNA
aactcaggttaggtactgaactcatca

(19) United States
(12) Patent Application Publication
(10) Pub. No.: US 2025/0235555 A1
(43) Pub. Date: Jul. 24, 2025

(54) METHODS FOR INTRATUMORAL DELIVERY OF CRISPR/CAS SYSTEMS

(71) Applicant: Christiana Care Gene Editing Institute, Inc., Wilmington, DE (US)

(72) Inventors: Eric B. Kmiec, Middletown, DE (US); Byung-Chun Yoo, Newark, DE (US); Steven Yang, Chapel Hill, NC (US); Kelly H. Banas, Bear, DE (US)

(73) Assignee: Christiana Care Gene Editing Institute, Inc., Wilmington, DE (US)

(21) Appl. No.: 18/711,261

(22) PCT Filed: Nov. 18, 2022
(86) PCT No.: PCT/US2022/050465

§ 371 Date: May 17, 2024

Related U.S. Application Data
(60) Provisional application No. 63/281,361, filed on Nov. 19, 2021.

(51) Int. Cl. A61K 48/00; A61K 38/46; A61P 35/00; C12N 9/22; C12N 15/11; C12N 15/86; C12N 15/88; C12N 15/90

(52) U.S. Cl. CPC A61K 48/005; A61K 38/465; A61P 35/00; C12N 9/22; C12N 15/11; C12N 15/86; C12N 15/88; C12N 15/907; C12N 2310/20; C12N 2710/10043; C12N 2750/14143

(57) ABSTRACT

This disclosure relates to methods treating a solid tumor by intratumorally administering a CRISPR/Cas system comprising one or more nucleic acid sequences encoding one or more guide RNAs that are complementary to one or more target sequences in a cancer gene of a cancer cell of the solid tumor and a nucleic acid sequence encoding a CRISPR-associated endonuclease.

### CROSS-REFERENCE TO RELATED APPLICATIONS

The present application claims priority under 35 U.S.C. § 119(e) to U.S. Provisional Patent Application No. 63/281,361, filed Nov. 19, 2021, which is incorporated herein by reference in its entirety.

### SEQUENCE LISTING

The Sequence Listing associated with this application is filed in electronic format and is hereby incorporated by reference into the specification in its entirety. The name of the text file containing the Sequence Listing is 13094901120 sequencelisting. The size of the text file is 39 kb, and the text file was created on Nov. 17, 2022.

### FIELD

The field relates to treatment of cancer through intratumoral delivery of CRISPR/Cas systems.

### BACKGROUND

Conventional systemic cancer chemotherapy and immunotherapy has severely limited the safety and effectiveness of such therapies because of toxicity issues. Additionally, patient quality of life for patients may be seriously compromised by such therapies. Intratumoral therapy has been considered as an alternative to systemic chemotherapy or immunotherapy, but during the past three decades, intratumoral therapy has been discouraged. There are at least three main reasons around such discouragement of intratumoral therapy: the tumor can be resected and therefore direct treatment is unnecessary, a direct injection will stimulate metastases, and local targeting provides no benefit in dealing with metastases (Goldberg et al., J. Pharm. Pharmacol. 54:159-80 (2002)).

CRISPR/Cas systems can be delivered to cells in several formats including plasmid DNA encoding the CRISPR-associated protein and guide RNA, and mRNA encoding the CRISPR-associated protein and guide RNA as synthetic molecules that can include chemically modified bases to enhance activity and stability and reduce toxicity. CRISPR-associated proteins and guide RNAs can be delivered as a preassembled ribonucleoprotein (RNP) complex. The benefit of preassembled RNP delivery over plasmid or mRNA are that there is no need to transcribe RNA or translate protein to elicit editing, which can maximize efficiency, and a shorter term of nuclease activity due to RNP decay leading to greater safety and therefore fewer off-target effects.

One of the major challenges for effective cancer therapy is ability to deliver a CRISPR/Cas system to a solid tumor. There thus remains a need to improve delivery of CRISPR/Cas systems to solid tumors.

### SUMMARY

One aspect is for a method of treating a solid tumor comprising intratumorally or peritumorally administering to a subject in need thereof a therapeutically effective amount of a CRISPR/Cas system comprising (a) one or more nucleic acid sequences encoding one or more guide RNAs (gRNAs) that are complementary to one or more target sequences in a target cell and (b) a nucleic acid sequence encoding a CRISPR-associated endonuclease.

In some embodiments, the one or more gRNAs comprise a trans-activated small RNA (tracrRNA) and a CRISPR RNA (crRNA).

In some embodiments, the one or more gRNAs are one or more single guide RNAs.

In some embodiments, the target cell is a cancer cell of the solid tumor; in some embodiments, the one or more target sequences is a cancer gene; in some embodiments, the cancer gene is NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNX1T1, DNMT3A, SMC1A, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPM1D, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF21, RIT1, MGA, ABL1, BRAF, CHEK1, FANCC, JAK2, MITF, PDCD1LG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSF1R, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGF1R, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, or STAT3; and in some embodiments, the cancer gene is NRF2 or EGFR.

In some embodiments, the CRISPR-associated endonuclease is a class 2 CRISPR-associated endonuclease; and in some embodiments, the class 2 CRISPR-associated endonuclease is Cas9 or Cas12a.

In some embodiments, the CRISPR/Cas system is comprised in a ribonucleoprotein (RNP) or lipid nanoparticle (LNP) complex.

In some embodiments, the one or more vectors driving expression of one or more elements of the CRISPR system are administered to the subject; in some embodiments, the one or more vectors is a viral vector, a liposome, or a lipid-containing complex; and in some embodiments, the viral vector is an adenovirus, an adenovirus-associated virus (AAV), a helper-dependent adenovirus, a retrovirus, or a hemagglutinating virus of Japan-liposome (HVJ) complex.

In some embodiments, the solid tumor is an adenoid cystic carcinoma tumor, a biliary tract cancer tumor, a bladder cancer tumor, a bone cancer tumor, a breast cancer tumor, a cervical cancer tumor, a cholangiocarcinoma tumor, a colon cancer tumor, an endometrial cancer tumor, an esophageal cancer tumor, a gallbladder cancer tumor, a gastric cancer tumor, a head and neck cancer tumor, a hepatocellular cancer tumor, a kidney cancer tumor, a lip cancer tumor, a liver cancer tumor, a melanoma tumor, a mesothelioma tumor, a non-small cell lung cancer tumor, a nonmelanoma skin cancer tumor, an oral cancer tumor, an ovarian cancer tumor, a pancreatic cancer tumor, a prostate cancer tumor, a rectal cancer tumor, a renal cancer tumor, a sarcoma tumor, a small cell lung cancer tumor, a spleen cancer tumor, a thyroid cancer tumor, a urothelial cancer tumor, or a uterine cancer tumor.

Another aspect is for a method of reducing expression of a cancer gene in a cancer cell of a solid tumor comprising intratumorally or peritumorally introducing into the cancer cell (a) one or more nucleic acid sequences encoding one or more guide RNAs (gRNAs) that are complementary to one or more target sequences in the cancer gene and (b) a nucleic acid sequence encoding a CRISPR-associated endonuclease, whereby the one or more gRNAs hybridize to the cancer gene and the CRISPR-associated endonuclease cleaves the cancer gene.

In some embodiments, the one or more gRNAs comprise a trans-activated small RNA (tracrRNA) and a CRISPR RNA (crRNA).

In some embodiments, the one or more gRNAs are one or more single guide RNAs.

In some embodiments, wherein the CRISPR-associated endonuclease is a class 2 CRISPR-associated endonuclease; and in some embodiments, the class 2 CRISPR-associated endonuclease is Cas9 or Cas12a.

In some embodiments, activity of the cancer gene is reduced in the cancer cell; in some embodiments, expression or activity of the cancer gene is not completely eliminated in the cancer cell; and in some embodiments, expression or activity of the cancer gene is completely eliminated in the cancer cell.

In some embodiments, the one or more nucleic acid sequences of (a) and the nucleic acid sequence of (b) is comprised in an RNP or LNP complex.

In some embodiments, the one or more vectors driving expression of one or more elements of the CRISPR system are administered to the subject; in some embodiments, the one or more vectors is a viral vector, a liposome, or a lipid-containing complex; in some embodiments, the viral vector is an adenovirus, an AAV, a helper-dependent adenovirus, a retrovirus, or a hemagglutinating virus of HVJ complex.

In some embodiments, the solid tumor is an adenoid cystic carcinoma tumor, a biliary tract cancer tumor, a bladder cancer tumor, a bone cancer tumor, a breast cancer tumor, a cervical cancer tumor, a cholangiocarcinoma tumor, a colon cancer tumor, an endometrial cancer tumor, an esophageal cancer tumor, a gallbladder cancer tumor, a gastric cancer tumor, a head and neck cancer tumor, a hepatocellular cancer tumor, a kidney cancer tumor, a lip cancer tumor, a liver cancer tumor, a melanoma tumor, a mesothelioma tumor, a non-small cell lung cancer tumor, a nonmelanoma skin cancer tumor, an oral cancer tumor, an ovarian cancer tumor, a pancreatic cancer tumor, a prostate cancer tumor, a rectal cancer tumor, a renal cancer tumor, a sarcoma tumor, a small cell lung cancer tumor, a spleen cancer tumor, a thyroid cancer tumor, a urothelial cancer tumor, or a uterine cancer tumor.

In some embodiments, the cancer gene is NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNX1T1, DNMT3A, SMC1A, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPM1D, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF21, RIT1, MGA, ABL1, BRAF, CHEK1, FANCC, JAK2, MITF, PDCD1LG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSF1R, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGF1R, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, or STAT3; and in some embodiments, the cancer gene is NRF2 or EGFR.

In some embodiments, intratumoral or peritumoral delivery of the CRISPR/Cas system results in at least about a 20% reduction in tumor size as compared to an untreated tumor.

In some embodiments, intratumoral or peritumoral delivery of the CRISPR/Cas system results in at least about a 20% inhibition in tumor growth as compared to an untreated tumor.

In some embodiments, the method further comprises administering one or more chemotherapeutic agents to the subject; and in some embodiments, intratumoral or peritumoral delivery of the CRISPR/Cas system reduces the amount of one or more chemotherapeutic agents administered to the subject as compared to a subject that does not receive administration of the CRISPR/Cas system.

Other objects and advantages will become apparent to those skilled in the art upon reference to the detailed description that hereinafter follows.

### BRIEF DESCRIPTION OF THE DRAWINGS

FIG. 1 shows exemplary NRF2 R34G sgRNA adenoviral vector constructs for evaluation of different sgRNA copy numbers and dual vector systems.

FIG. 2 shows the % indel formation after adenoviral vector mediated gene editing in lung cancer cell lines C26-8 and C44-25 treated with NRF2 R34G sgRNAs.

FIG. 3 shows a comparison of the transduction efficacy in lung cancer cells of AAV and adenovirus as determined by GFP expression. The numbers right above the scale bars represented the percentage of GFP positive cells, and the numbers at the left corners of each image indicated the mean fluorescent intensity (MFI) of GFP.

FIG. 4 shows a comparison of AAV6 and adenovirus for expression of Cas9 in lung cancer cells, as indicated by editing efficacy of an NRF2 R34G gRNA.

FIG. 5 Intratumoral injection of LNP results in high luciferase expression in tumor samples and minimal biodistribution. qPCR analysis of luciferase expression normalized to Gapdh in tissues from NCG mice injected with LNP (n=5 per LNP). Error bars show +/−standard deviation.

FIG. 6 Bioluminescent imaging of athymic nude mice shows expression of luciferase in the area of intratumoral injection at 4 and 24 hr. A. In vivo imaging of athymic nude mice injected with LNP at 4 and 24 hrs. B. Quantification of in vivo imaging at 4 and 24 hrs.

FIG. 7 Intratumoral delivery of adenoviral vector (fLuc) in H1703 44-25 subcutaneous xenograft model. Mice were intratumorally injected with Ad-CMV-fLuc and imaged 1-16 days post injection. The bioluminescent signal from each tumor was quantified by establishing a ROI and each value listed is total flux photons. The scale bars are representative for each image to the left of the bar.

FIG. 8 Schematic diagram of CRISPR/Cas9 adenoviral vectors targeting NRF2. The diagram depicts the U6 promoter driving R34G-targeting sgRNA expression followed by the chicken beta actin (CAG) promoter driving enhanced SpCas9 expression.

FIG. 9 Representative images of Cas9 immunostaining of H1703 44-25-derived tumors. The two left hand panels depict two magnifications, 5× and 20×, from the same tumor section of an adenovirus-treated tumor. The scale bars represent 200 μm and 50 μm, respectively. The two right hand panels depict two magnifications, 2.5× and 20×, from the same tumor section of an untreated tumor. The scale bars represent 500 μm and 50 μm, respectively.

FIG. 10 Representative images of Cas9 immunostaining of PDX-derived tumors. The two left hand panels depict two magnifications, 5× and 20×, from the same tumor section of an adenovirus-treated tumor. The scale bars represent 200 μm and 50 μm, respectively. The two right hand panels depict two magnifications, 2.5× and 20×, from the same tumor section of an untreated tumor. The scale bars represent 500 μm and 50 μm, respectively.

FIG. 11 Schematic diagram of CRISPR/Cas9 adenoviral vectors targeting NRF2. A) The diagram depicts the U6, H1 and 7SK promoters driving R34G-targeting sgRNA (SEQ ID NO:25) expression followed by the chicken beta actin (CAG) promoter driving enhanced SpCas9 expression. B) The diagram depicts the U6, H1 and 7SK promoters driving scrambled-targeting sgRNA (SEQ ID NO:26) expression followed by the chicken beta actin (CAG) promoter driving enhanced SpCas9 expression.

FIG. 12 Delivery of CRISPR/Cas9 adenovirus in combination with chemotherapy inhibits tumor growth in a xenograft model. The graph shows tumor growth in NRF2-targeted or scramble-targeted mice over the course of 22 days. Mice were intratumorally injected on days 0, 2, and 7 with 3.6e⁹ pfu of either Ad-U6H17SK-R34G-CAG-eSpCas9 or U6H17SK-scramble-CAG-eSpCas9, as denoted by \hat{ } along the x axis. On days 3 and 10, all mice received 12.5 mg/kg of carboplatin and 5 mg/kg paclitaxel, intravenously, as denoted by * along the x axis.

FIG. 13 Genomic analyses of NRF2-targeted tumor tissues from xenograft mouse models. A) A single injection of 3.6e⁹ pfu of Ad-U6-R34G-CAG-eSpCas9 was intratumorally delivered to a patient-derived xenograft model. B) Two injections of 3.6e⁹ pfu of Ad-U6H17SK-R34G-CAG-eSpCas9 was intratumorally delivered to a H1703 44-25 xenograft model. C) Three injections of 3.6e⁹ pfu of Ad-U6H17SK-R34G-CAG-eSpCas9 was intratumorally delivered to a H1703 44-25 xenograft model. Tumor tissues from respective mouse models were excised and analyzed by PCR and Sanger sequencing.

FIG. 14 Intratumoral delivery of luciferase-containing lipid nanoparticles in H1703 44-25 subcutaneous xenograft model. Mice were intratumorally injected with lipid nanoparticles and imaged 4 and 24 hours post injection. The bioluminescent signal from each tumor was quantified by establishing a ROI and each value listed is total flux photons. The scale is representative of all images.

FIG. 15 Intratumoral delivery of luciferase-containing lipid nanoparticles in a Patient-derived xenograft model. Mice were intratumorally injected with lipid nanoparticles and imaged 4 and 24 hours post injection. The bioluminescent signal from each tumor was quantified by establishing a ROI and each value listed is total flux photons. The scale is representative of all images.

FIG. 16 Intratumoral delivery of luciferase-containing lipid nanoparticles in a Patient-derived xenograft model. Mice were intratumorally injected with lipid nanoparticles and imaged 4 and 24 hours post injection. The bioluminescent signal from each tumor was quantified by establishing a ROI and each value listed is total flux photons. The scale is representative of all images.

FIG. 17 shows in vivo AAV tropism assessment for AAV5 and AAV6 containing a luciferase gene in mice implanted with H1703 squamous non-small cell lung carcinoma cells. The representative median animal was chosen as the one with a flux value closest to the median of all the surviving animals at the last time point at which at least 50% of the animals remain in the group.

FIG. 18 shows tumor volume (mm³) and in vivo bioluminescence (Flux, pho/sec) over time for a representative mouse implanted with H1703 squamous non-small cell lung carcinoma cells and treated intratumorally with AAV6 containing a luciferase gene.

FIG. 19 shows the biodistribution of AAV6 containing a luciferase gene (AAV6-fLuc) injected into tumors of mice implanted with H1703 squamous non-small cell lung carcinoma cells. Ex-vivo bioluminescence was measured at Day 21. Animals were implanted subcutaneously with 5×106 H1703 cells. After tumor volume reached >60 mm3, AAV6-fLuc was injected into the tumor directly and luciferase expression was observed by ex vivo imaging of isolated tissues. Data are shown unscaled.

### DETAILED DESCRIPTION

Applicant specifically incorporates the entire contents of all cited references in this disclosure. Further, when an amount, concentration, or other value or parameter is given as either a range or a list of upper values and lower values, this is to be understood as specifically disclosing all ranges formed from any pair of any upper range limit or value and any lower range limit or value, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

### Definitions

In this disclosure, a number of terms and abbreviations are used. The following definitions are provided.

As used herein, the term “about” or “approximately” means within 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or less of a given value or range.

The term “comprising” is intended to include embodiments encompassed by the terms “consisting essentially of” and “consisting of”. Similarly, the term “consisting essentially of” is intended to include embodiments encompassed by the term “consisting of”.

The indefinite articles “a” and “an”, as used herein in the specification and in the claims, unless clearly indicated to the contrary, should be understood to mean “at least one”.

The phrase “and/or”, as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, “either”, “one of”, “only one of”, “exactly one of”. “Consisting essentially of”, when used in the claims, shall have its ordinary meaning as used in the field of patent law.

An “endonuclease” an enzyme that cleaves the phosphodiester bond within a polynucleotide chain. In some embodiments, an endonuclease generates a double-stranded break at a desired position in the genome, and in some embodiments, an endonuclease generates a single-stranded break or a “nick” or break on one strand of the DNA phosphate sugar backbone at a desired position in the genome, and in some embodiments, without producing undesired off-target DNA stranded breaks. Endonuclease can be naturally occurring endonuclease or it can be artificially generated.

A “Clustered Regularly Interspaced Short Palindromic Repeat (CRISPR)-associated endonuclease protein-binding domain” or “Cas binding domain” refers to a nucleic acid element or domain within a nucleic acid sequence or polynucleotide sequence that, in an effective amount, will bind or have an affinity for one or a plurality of CRISPR-associated endonuclease (or functional fragments thereof). In some embodiments, in the presence of the one or a plurality of proteins (or functional fragments thereof) and a target sequence, the one or plurality of proteins and the nucleic acid element forms a biologically active CRISPR complex and/or can be enzymatically active on a target sequence. In some embodiments, the CRISPR-associated endonuclease is a class 1 or class 2 CRISPR-associated endonuclease, and in some embodiments, a Cas9 or Cas12a endonuclease. The Cas9 endonuclease can have a nucleotide sequence identical to the wild type Streptococcus pyogenes sequence. In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Such species include: Acidovorax avenae, Actinobacillus pleuropneumoniae, Actinobacillus succinogenes, Actinobacillus suis, Actinomyces sp., Alicycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., and Verminephrobacter eiseniae (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas9 endonucleases). In some embodiments, the CRISPR-associated endonuclease can be a Cas12a nuclease. The Cas12a nuclease can have a nucleotide sequence identical to a wild type Prevotella, Francisella, Acidaminococcus, Proteocatella, Sulfurimonas, Elizabethkingia, Methylococcales, Moraxella, Helcococcus, Lachnospira, Limihaloglobus, Butyrivibrio, Methanomethylophilus, Coprococcus, Synergistes, Eubacterium, Roseburia, Bacteroidales, Ruminococcus, Eubacteriaceae, Leptospira, Parabacteriodes, Gracilibacteria, Lachnospiraceae, Clostridium, Brumimicrobium, Fibrobacter, Catenovulum, Acinetobacter, Flavobacterium, Succiniclasticum, Pseudobutyrivibrio, Barnesiella, Sneathia, Succinivibrionaceae, Treponema, Sedimentisphaera, Thiomicrospira, Eucomonympha, Arcobacter, Oribacterium, Methanoplasma, Porphyromonas, Succinovibrio, or Anaerovibrio sequence (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas12a endonucleases).

In some embodiments, the terms “(CRISPR)-associated endonuclease protein-binding domain” or “Cas binding domain” refer to a nucleic acid element or domain (e.g. and RNA element or domain) within a nucleic acid sequence that, in an effective amount, will bind to or have an affinity for one or a plurality of CRISPR-associated endonucleases (or functional fragments or variants thereof that are at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to a CRISPR-associated endonuclease). In some embodiments, the Cas binding domain consists of at least or no more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially associates or binds to a biologically active CRISPR-associated endonuclease at a concentration and within a microenvironment suitable for CRISPR system formation.

The “Clustered Regularly Interspaced Short Palindromic Repeats (CRISPR)-CRISPR associated (Cas) (CRISPR-Cas) system guide RNA” or “CRISPR-Cas system guide RNA” may comprise a transcription terminator domain. The term “transcription terminator domain” refers to a nucleic acid element or domain within a nucleic acid sequence (or polynucleotide sequence) that, in an effective amount, prevents bacterial transcription when the CRISPR complex is in a bacterial species and/or creates a secondary structure that stabilizes the association of the nucleic acid sequence to one or a plurality of Cas proteins (or functional fragments thereof) such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of at least or no more than about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially drives association of the nucleic acid sequence (sgRNA, crRNA with tracrRNA, or other nucleic acid sequence) to a biologically active CRISPR complex at a concentration and microenvironment suitable for CRISPR complex formation.

The term “DNA-binding domain” refers to a nucleic acid element or domain within a nucleic acid sequence (e.g. a guide RNA) that is complementary to a target sequence. In some embodiments, the DNA-binding domain will bind or have an affinity for a target sequence such that, in the presence of a biologically active CRISPR complex, one or plurality of Cas proteins can be enzymatically active on the target sequence. In some embodiments, the DNA binding domain comprises at least one sequence that is capable of forming Watson Crick basepairs with a target sequence as part of a biologically active CRISPR system at a concentration and microenvironment suitable for CRISPR system formation.

“CRISPR system” or “CRISPR/Cas system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex.

A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is an guanine and N is any naturally occurring nucleic acid. In some embodiments the target DNA comprises any one or combination of NNG, NNA, GAA, NGGNG, NGRRT, NGRRN, NNNNGATT, NNNNRYAC, NNAGAAW, TTTV, YG, TTTN, YTN, NGCG, NGAG, NGAN, NGNG, NG, NNGRRT, TYCV, TATV, or NAAAAC. In some embodiments, a target sequence is located in the nucleus or cytoplasm of a cell.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more base pairs from) the target sequence. Without wishing to be bound by theory, the tracr sequence, which may comprise or consist of all or a portion of a wild-type tracr sequence (e.g., about or more than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a class 2 Cas9 or Cas12a protein. In some embodiments, the chemically synthesized guide sequence is used in conjunction with one or more vectors, wherein each vector comprises a coding sequence that encodes a CRISPR enzyme, such as a class 2 Cas9 or Cas12a protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, one or more additional guide sequence, tracr mate sequence, and tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid for a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

In some embodiments, a guide RNA can be a short, synthetic, chimeric tracrRNA/crRNA (a “single-guide RNA” or “sgRNA”). A guide RNA may also comprise two short, synthetic tracrRNA/crRNAs (a “dual-guide RNA” or “dgRNA”).

As used herein, the term “homologous” or “homologue” or “ortholog” refers to related sequences that share a common ancestor or family member and are determined based on the degree of sequence identity. The terms “homology”, “homologous”, “substantially similar”, and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. In some embodiments, these terms describe the relationship between a gene found in one species, subspecies, variety, cultivar, or strain and the corresponding or equivalent gene in another species, subspecies, variety, cultivar or strain. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania), AlignX (Vector NTI, Invitrogen, Carlsbad, Calif.), and Sequencher (Gene Codes, Ann Arbor, Mich.).

By “hybridizable”, “complementary”, or “substantially complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize”, to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C). In addition, for hybridization between two RNA molecules (e.g., dsRNA), and for hybridization of a DNA molecule with an RNA molecule (e.g., when a ssRNA target nucleic acid base pairs with a DNA PAMmer, when a DNA target nucleic acid base pairs with an RNA guide nucleic acid, etc.): guanine (G) can also base pair with uracil (U). For example, G/U base-pairing is partially responsible for the degeneracy (i.e., redundancy) of the genetic code in the context of tRNA anti-codon base-pairing with codons in mRNA. Thus, a guanine (G) (e.g., of a protein-binding segment (dsRNA duplex) of a subject guide nucleic acid molecule, of a target nucleic acid base pairing with a guide nucleic acid, and/or a PAMmer, etc.) is considered complementary to both a uracil (U) and to an adenine (A). For example, when a G/U base-pair can be made at a given nucleotide position of a protein-binding segment (e.g., dsRNA duplex) of a subject guide nucleic acid molecule, the position is not considered to be non-complementary, but is instead considered to be complementary.

Hybridization and washing conditions are well known and exemplified in Sambrook J., Fritsch. E. F. and Maniatis, T. Molecular Cloning: A Laboratory Manual, Second Edition. Cold Spring Harbor Laboratory Press. Cold Spring Harbor (1989), particularly Chapter 11 and Table 11.1 therein; and Sambrook. J. and Russell, W., Molecular Cloning: A Laboratory Manual, Third Edition. Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g., complementarity over 35, 34, 33, 32, 31, 30, 29, 28, 27, 26, 25, 24, 23, 22, 21, 20, 19, 18 or less nucleotides), the position of mismatches can become important (see Sambrook et al., supra, 11.7-11.8). Typically, the length for a hybridizable nucleic acid is 8 nucleotides or more (e.g., 10, 11, 12, 13, 14, 15, 16, 17, 18, 19, 20, 21, 22, 23, 24, 25, 26, 27, 28, 29, 30 nucleotides or more). The temperature and wash solution salt concentration may be adjusted as necessary according to factors such as length of the region of complementation and the degree of complementation.

Examples of stringent hybridization conditions include: incubation temperatures of about 25° C., 26° C., 27° C., 28° C., 29° C., 30° C., 31° C., 32° C., 33° C., 34° C., 35° C., 36° C., or 37° C.; hybridization buffer concentrations of about 6×SSC, 7×SSC, 8×SSC, 9×SSC, or 10×SSC; formamide concentrations of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%; and wash solutions from about 4×SSC, 5×SSC, 6×SSC, 7×SSC, to 8×SSC. Examples of moderate hybridization conditions include: incubation temperatures of about 40° C., 41° C., 42° C., 43° C., 44° C., 45° C., 46° C., 47° C., 48° C., 49° C., or 50° C.; buffer concentrations of about 9×SSC, 8×SSC, 7×SSC, 6×SSC, 5×SSC, 4×SSC, 3×SSC, or 2×SSC; formamide concentrations of about 30%, 31%, 32%, 33%, 34%, 35%, 36%, 37%, 38%, 39%, 40%, 41%, 42%, 43%, 44%, 45%, 46%, 47%, 48%, 49%, or 50%; and wash solutions of about 5×SSC, 4×SSC, 3×SSC, or 2×SSC. Examples of high stringency conditions include: incubation temperatures of about 55%, 56%, 57%, 58%, 59%, 60%, 61%, 62%, 63%, 64%, 65%, 66%, 67%, or 68%; buffer concentrations of about 1×SSC, 0.95×SSC, 0.9×SSC, 0.85×SSC, 0.8×SSC, 0.75×SSC, 0.7×SSC, 0.65×SSC, 0.6×SSC, 0.55×SSC, 0.5×SSC, 0.45×SSC, 0.4×SSC, 0.35×SSC, 0.3×SSC, 0.25×SSC, 0.2×SSC, 0.15×SSC, or 0.1×SSC; formamide concentrations of about 55%, 56%, 57%, 58%, 59%, 60%, 61%, 62%, 63%, 64%, 65%, 66%, 67%, 68%, 69%, 70%, 71%, 72%, 73%, 74%, or 75%; and wash solutions of about 1×SSC, 0.95×SSC, 0.9×SSC, 0.85×SSC, 0.8×SSC, 0.75×SSC, 0.7×SSC, 0.65×SSC, 0.6×SSC, 0.55×SSC, 0.5×SSC, 0.45×SSC, 0.4×SSC, 0.35×SSC, 0.3×SSC, 0.25×SSC, 0.2×SSC, 0.15×SSC, or 0.1×SSC, or deionized water. In general, hybridization incubation times are from 5 minutes to 24 hours, with 1, 2, or more washing steps, and wash incubation times are about 1, 2, 3, 4, 5, 6, 7, 8, 9, 10, 11, 12, 13, 14, 15 minutes or more. It is understood that equivalents of SSC using other buffer systems can be employed.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% (i.e., full complementarity) sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90% complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Exemplary methods include BLAST programs (basic local alignment search tools) and PowerBLAST programs (Altschul et al., J. Mol. Biol. 215:403-10 (1990); Zhang et al., Genome Res., 7:649-56 (1997)) or by using the Gap program (Wisconsin Sequence Analysis Package, Version 8 for Unix, Genetics Computer Group, University Research Park, Madison Wis.), using default settings, which uses the algorithm of Smith et al. (Adv. Appl. Math. 2:482-89 (1981)).

The term “intratumoral”, as used herein, refers to delivery or transport of a CRISPR/Cas system into a tumor. One example of intratumoral delivery, or transport, of a CRISPR/Cas system as described herein is by intratumoral administration, a route of administration generally known in the art. As an alternative route for intratumoral administration, a CRISPR/Cas system may be delivered to the tumor via a tumor-specific carrier, such as an oncolytic virus or a gene therapy vector, which have been broadly developed to deliver gene sequences to tumors. In some embodiments, delivery or transport to a tumor may include delivery or transport of a CRISPR/Cas system on the periphery of the solid tumor (“peritumorally”), such as if the amount of a CRISPR/Cas system thereof is too large to all be directly delivered or transported into the solid tumor, or if treatment of the tumor can be more effectively accomplished by delivery or transport of a CRISPR/Cas system to the periphery of the tumor, or e.g. anywhere generally within about a 2.5 cm thick zone surrounding the margins of a tumor (e.g., in an organ, e.g., the brain, lung, liver, bladder, kidney, stomach, intestines, breast, pancrease, prostate, oviary, esophagus, spleen, thyroid). Multiple injections into separate regions of the tumor or cancer are also included. Furthermore, intratumoral administration includes delivery of a composition into one or more metastases.

As used herein, a “solid tumor” is an abnormal mass of tissue that usually does not contain cysts or liquid areas. Solid tumors may be benign or malignant. Different types of solid tumors are named for the type of cells that form them. In some embodiments, the solid tumor is an adenoid cystic carcinoma tumor, a biliary tract cancer tumor, a bladder cancer tumor, a bone cancer tumor, a breast cancer tumor, a cervical cancer tumor, a cholangiocarcinoma tumor, a colon cancer tumor, an endometrial cancer tumor, an esophageal cancer tumor, a gallbladder cancer tumor, a gastric cancer tumor, a head and neck cancer tumor, a hepatocellular cancer tumor, a kidney cancer tumor, a lip cancer tumor, a liver cancer tumor, a melanoma tumor, a mesothelioma tumor, a non-small cell lung cancer tumor, a nonmelanoma skin cancer tumor, an oral cancer tumor, an ovarian cancer tumor, a pancreatic cancer tumor, a prostate cancer tumor, a rectal cancer tumor, a renal cancer tumor, a sarcoma tumor, a small cell lung cancer tumor, a spleen cancer tumor, a thyroid cancer tumor, a urothelial cancer tumor, or a uterine cancer tumor. In some embodiments, the solid tumor is a benign tumor, and the subject has cancer elsewhere in the body. In some embodiments, the solid tumor is a malignant solid tumor. In some embodiments, the malignant solid tumor is the only cancer in the body of the subject. In other embodiments, the subject has a malignant solid tumor and cancer in other areas of the body.

As used herein, a “variant”, “mutant”, or “mutated” polynucleotide contains at least one polynucleotide sequence alteration as compared to the polynucleotide sequence of the corresponding wild-type or parent polynucleotide.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

### CRISPR/Endonucleases

CRISPR/endonuclease (e.g., CRISPR/Cas9) systems are known in the art and are described, for example, in U.S. Pat. No. 9,925,248, which is incorporated by reference herein in its entirety. CRISPR-directed gene editing can identify and execute DNA cleavage at specific sites within the chromosome at a surprisingly high efficiency and precision. The natural activity of CRISPR/Cas9 is to disable a viral genome infecting a bacterial cell. Subsequent genetic reengineering of CRISPR/Cas function in human cells presents the possibility of disabling human genes at a significant frequency.

In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA) containing a DNA binding region (spacer) which is complementary to the target gene.

The compositions described herein can include a nucleic acid encoding a CRISPR-associated endonuclease. The CRISPR-associated endonuclease can be, e.g., a class 1 CRISPR-associated endonuclease or a class 2 CRISPR-associated endonuclease. Class 1 CRISPR-associated endonucleases include type I, type III, and type IV CRISPR-Cas systems, which have effector molecules that comprise multiple subunits. For class 1 CRISPR-associated endonucleases, effector molecules can include, in some embodiments, Cas7 and Cas5, along with, in some embodiments, SS (Cas11) and Cas8a1; Cas8b1; Cas8c; Cas8u2 and Cas6; Cas3″ and Cas10d; Cas SS (Cas11), Cas8e, and Cas6; Cas8f and Cas6f; Cas6f; Cas8-like (Csf1); SS (Cas11) and Cas8-like (Csf1); or SS (Cas11) and Cas10. Class 1 CRISPR-associated endonucleases also be associated with, in some embodiments, target cleavage molecules, which can be Cas3 (type I) or Cas10 (type III) and spacer acquisition molecules such as, e.g., Cas1, Cas2, and/or Cas4. See, e.g., Koonin et al., Curr. Opin. Microbiol. 37:67-78 (2017); Strich et al., J. Clin. Microbiol. 57:1307-18 (2019).

Class 2 CRISPR-associated endonucleases include type I, type V, and type VI CRISPR-Cas systems, which have a single effector molecule. For class 2 CRISPR-associated endonucleases, effector molecules can include, in some embodiments, Cas9, Cas12a (cpf1), Cas12b1 (c2c1), Cas12b2, Cas12c (c2c3), Cas12d (CasY), Cas12e (CasX), Cas12f1 (Cas14a), Cas12f2 (Cas14b), Cas12f3 (Cas14c), Cas12g, Cas12h, Cas12i, Cas12k (c2c5), Cas12j (Cas@), Cas13a (c2c2), Cas13b1 (c2c6), Cas13b2 (c2c6), Cas13c (c2c7), Cas13d, c2c4, c2c8, c2c9, and/or c2c10. See, e.g., Koonin et al., Curr. Opin. Microbiol. 37:67-78 (2017); Strich et al., J. Clin. Microbiol. 57:1307-18 (2019); Makarova et al., Nat. Rev. Microbiol. 18:67-83 (2020); Pausch et al., Science 369:333-37 (2020).

In some embodiments, the CRISPR-associated endonuclease can be a Cas9 nuclease. The Cas9 nuclease can have a nucleotide sequence identical to the wild type Streptococcus pyogenes sequence. In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Such species include: Acidovorax avenae, Actinobacillus pleuropneumoniae, Actinobacillus succinogenes, Actinobacillus suis, Actinomyces sp., Alicycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., and Verminephrobacter eiseniae.

Alternatively, the wild type Streptococcus pyogenes Cas9 sequence can be modified. The nucleic acid sequence can be codon optimized for efficient expression in mammalian cells, e.g., human cells. A Cas9 nuclease sequence codon optimized for expression in human cells sequence can be for example, the Cas9 nuclease sequence encoded by any of the expression vectors listed in Genbank accession numbers KM099231.1 GI: 669193757; KM099232.1 GI: 669193761; or KM099233.1 GI: 669193765. Alternatively, the Cas9 nuclease sequence can be, for example, the sequence contained within a commercially available vector such as pX458, pX330 or pX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI: 669193757; KM099232.1 GI: 669193761; or KM099233.1 GI: 669193765 or Cas9 amino acid sequence of pX458, pX330 or pX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more, e.g., insertions, deletions, or mutations or a combination thereof. One or more of the mutations can be a substitution (e.g., a conservative amino acid substitution). For example, a biologically active variant of a Cas9 polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas9 polypeptide.

In some embodiments, the CRISPR-associated endonuclease can be a Cas12a nuclease. The Cas12a nuclease can have a nucleotide sequence identical to a wild type Prevotella or Francisella sequence. Alternatively, a wild type Prevotella or Francisella Cas12a sequence can be modified. In some embodiments, an Acidaminococcus, Proteocatella, Sulfurimonas, Elizabethkingia, Methylococcales, Moraxella, Helcococcus, Lachnospira, Limihaloglobus, Butyrivibrio, Methanomethylophilus, Coprococcus, Synergistes, Eubacterium, Roseburia, Bacteroidales, Ruminococcus, Eubacteriaceae, Leptospira, Parabacteriodes, Gracilibacteria, Lachnospiraceae, Clostridium, Brumimicrobium, Fibrobacter, Catenovulum, Acinetobacter, Flavobacterium, Succiniclasticum, Pseudobutyrivibrio, Barnesiella, Sneathia, Succinivibrionaceae, Treponema, Sedimentisphaera, Thiomicrospira, Eucomonympha, Arcobacter, Oribacterium, Methanoplasma, Porphyromonas, Succinovibrio, or Anaerovibrio Cas12a sequence can be modified. The nucleic acid sequence can be codon optimized for efficient expression in mammalian cells, e.g., human cells. A Cas12a nuclease sequence codon optimized for expression in human cells sequence can be for example, the Cas9 nuclease sequence encoded by any of the expression vectors listed in Genbank accession numbers MF193599.1 GI: 1214941796, KY985374.1 GI: 1242863785, KY985375.1 GI: 1242863787, or KY985376.1 GI: 1242863789. Alternatively, the Cas12a nuclease sequence can be, for example, the sequence contained within a commercially available vector such as pAs-Cpf1 or pLb-Cpf1 from Addgene (Cambridge, Mass.). In some embodiments, the Cas12a endonuclease can have an amino acid sequence that is a variant or a fragment of any of the Cas12a endonuclease sequences of Genbank accession numbers MF193599.1 GI: 1214941796, KY985374.1 GI: 1242863785, KY985375.1 GI: 1242863787, or KY985376.1 GI: 1242863789 or Cas12a amino acid sequence of pAs-Cpf1 or pLb-Cpf1 (Addgene, Cambridge, Mass.). The Cas12a nucleotide sequence can be modified to encode biologically active variants of Cas12a, and these variants can have or can include, for example, an amino acid sequence that differs from a wild type Cas12a by virtue of containing one or more, e.g., insertions, deletions, or mutations or a combination thereof. One or more of the mutations can be a substitution (e.g., a conservative amino acid substitution). For example, a biologically active variant of a Cas12a polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas12a polypeptide.

The compositions described herein may also include sequence encoding a guide RNA (gRNA) comprising a DNA-binding domain that is complementary to a target domain in a target sequence, and a CRISPR-associated endonuclease protein-binding domain. The guide RNA sequence can be a sense or anti-sense sequence. The guide RNA sequence may include a PAM. The sequence of the PAM can vary depending upon the specificity requirements of the CRISPR endonuclease used. In, e.g., the CRISPR-Cas system derived from S. pyogenes, the target DNA typically immediately precedes a 5′-NGG proto-spacer adjacent motif (PAM). Thus, for the S. pyogenes Cas9, the PAM sequence can be NGG. Other Cas endonucleases may have different PAM specificities (e.g., NNG, NNA, GAA, NGGNG, NGRRT, NGRRN, NNNNGATT, NNNNRYAC, NNAGAAW, TTTV, YG, TTTN, YTN, NGCG, NGAG, NGAN, NGNG, NG, NNGRRT, TYCV, TATV, or NAAAAC). The specific sequence of the guide RNA may vary, but, regardless of the sequence, useful guide RNA sequences will be those that minimize off-target effects while achieving high efficiency.

In some embodiments, the DNA-binding domain varies in length from about 20 to about 55 nucleotides, for example, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides. In some embodiments, the Cas protein-binding domain is from about 30 to about 55 nucleotides in length, for example, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides.

In some embodiments, the compositions comprise one or more nucleic acid (i.e. DNA) sequences encoding the guide RNA and the CRISPR endonuclease. When the compositions are administered as a nucleic acid or are contained within an expression vector, the CRISPR endonuclease can be encoded by the same nucleic acid or vector as the guide RNA sequence. In some embodiments, the CRISPR endonuclease can be encoded in a physically separate nucleic acid from the guide RNA sequence or in a separate vector. The nucleic acid sequence encoding the guide RNA may comprise a DNA binding domain, a Cas protein binding domain, and a transcription terminator domain.

The nucleic acid encoding the guide RNA and/or the CRISPR endonuclease may be an isolated nucleic acid. An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids also can be obtained by mutagenesis of, e.g., a naturally occurring portion of a Cas9-encoding DNA (in accordance with, for example, the formula above).

Recombinant constructs are also provided herein and can be used to transform cells in order to express the CRISPR endonuclease and/or a guide RNA complementary to a target sequence. A recombinant nucleic acid construct may comprise a nucleic acid encoding a CRISPR endonuclease and/or a guide RNA complementary to a target sequence, operably linked to a promoter suitable for expressing the CRISPR endonuclease and/or a guide RNA complementary to the target sequence in the cell. In some embodiments, the nucleic acid encoding a CRISPR endonuclease is operably linked to the same promoter as the nucleic acid encoding the guide RNA. In other embodiments, the nucleic acid encoding a CRISPR endonuclease and the nucleic acid encoding the guide RNA are operably linked to different promoters. In some embodiments, the promoter can be one or more pol III promoters, one or more pol II promoters, one or more pol I promoters, or combinations thereof. Examples of pol III promoters include, but are not limited to, U6 and H1 promoters. Examples of pol II promoters include, but are not limited to, the retroviral Rous sarcoma virus (RSV), LTR promoter (optionally with the RSV enhancer), the cytomegalovirus (CMV) promoter (optionally with the CMV enhancer; see, e.g., Boshart et al., Cell 41:521-30 (1985)), the SV40 promoter, the dihydrofolate reductase promoter, the β-actin promoter, the phosphoglycerol kinase (PGK) promoter, and the EFla promoter. An example of a pol I promoter includes, but is not limited to, the 47S pre-rRNA promoter.

### Intratumoral Delivery

Any of the pharmaceutical compositions disclosed herein can be formulated for use in the preparation of a medicament, and particular uses are indicated below in the context of treatment, e.g., the treatment of a subject having cancer. When employed as pharmaceuticals, any of the nucleic acids and vectors can be administered in the form of pharmaceutical compositions. A subject is successfully “treated” according to the present methods if the subject shows one or more of the following: a reduction in tumorigenicity, a reduction in the number or frequency of cancer stem cells, an increased immune response, an increased anti-tumor response, increased cytolytic activity of immune cells, increased killing of tumor cells, increased killing of tumor cells by immune cells, a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; or some combination of effects.

In some embodiments, pharmaceutical compositions can contain, as the active ingredient, nucleic acids and vectors described herein in combination with one or more pharmaceutically acceptable carriers. The term “pharmaceutically acceptable” refers to molecular entities and compositions that do not produce an adverse, allergic or other untoward reaction when administered to an animal or a human, as appropriate. The term “pharmaceutically acceptable carrier”, as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the pharmaceutical compositions disclosed herein, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

Methods for intratumoral delivery of drugs are known in the art (Brincker, Crit. Rev. Oncol. Hematol. 15:91-98 (1993); Celikoglu et al., Cancer Ther. 6:545-52 (2008)). For example, a CRISPR/Cas system can be administered by conventional needle injection, needle-free jet injection or electroporation or combinations thereof into the tumor or cancer tissue. A CRISPR/Cas system can be administered directly into the tumor or cancer (tissue) with great precision using computer tomography, ultrasound, gamma camera imaging, positron emission tomography, or magnetic resonance tumor imaging. In some embodiments, CRISPR/Cas system intratumoral administration is by direct intratumoral administration by endoscopy, bronchoscopy, cystoscopy, colonoscopy, laparoscope, or catheterization. In some embodiments where the tumor is in contact with bodily fluid in a closed system, a CRISPR/Cas system may be administered to the fluid for intratumoral administration.

Intratumoral administration may be used to accomplish one or more of the following: reducing tumor size; reducing tumor growth; reducing or limiting development and/or spreading of metastases; eliminating a tumor; inhibiting, preventing, or reducing the recurrence of a tumor for at least 1, 2, 3, 4, 5, 6, 7, 8, 9, 10, 11, 12, or more months; and/or promoting an immune response against a tumor. The effect may be found in the tumor to which a CRISPR/Cas system was administered and/or one or more metastases or other tumor.

In some embodiments, one or more CRISPR endonucleases and one or more guide RNAs may be provided in combination in the form of ribonucleoprotein particles (RNPs). An RNP complex can be introduced into a subject by means of, e.g., injection, electroporation, nanoparticles (including, e.g., lipid nanoparticles), vesicles, and/or with the assistance of cell-penetrating peptides. See, e.g., Lin et al., ELife 3:e04766 (2014); Sansbury et al., CRISPR J. 2 (2): 121-32 (2019); US2019/0359973)

In some embodiments, one or more CRISPR endonuclease and one or more guide RNAs may be delivered by a lipid nanoparticle (LNP). An LNP refers to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm. LNPs may be made from cationic, anionic, or neutral lipids. Neutral lipids, such as the fusogenic phospholipid DOPE or the membrane component cholesterol, may be included in LNPs as “helper lipids” to enhance transfection activity and nanoparticle stability. LNPs may also be comprised of hydrophobic lipids, hydrophilic lipids, or both hydrophobic and hydrophilic lipids.

In certain embodiments, the cationic lipid N-[1-(2,3-dioleyloxy) propyl]-N,N,N-trimethylammonium chloride (DOTMA) can be used. DOTMA can be formulated alone or combined with the neutral lipid, dioleoylphosphatidyl-ethanolamine (DOPE) or other cationic or non-cationic lipids into a liposomal transfer vehicle or a lipid nanoparticle, and such liposomes can be used to enhance the delivery of nucleic acids into target cells. Other suitable cationic lipids include, but are not limited to, 5-carboxyspermylglycinedioctadecylamide, 2,3-dioleyloxy-N-[2(spermine-carboxamido)ethyl]-N,N-dimethyl-1-propanaminium, 1,2-Dioleoyl-3-Dimethylammonium-Propane, 1,2-Dioleoyl-3-Trimethylammonium-Propane. Contemplated cationic lipids also include 1,2-distearyloxy-N,N-dimethyl-3-aminopropane, 1,2-dioleyloxy-N,N-dimethyl-3-aminopropane, 1,2-dilinoleyloxy-N,N-dimethyl-3-aminopropane, 1,2-dilinolenyloxy-N,N-dimethyl-3-aminopropane, N-dioleyl-N,N-dimethylammonium chloride, N,N-distearyl-N,N-dimethylammonium bromide, N-(1,2-dimyristyloxyprop-3-yl)-N,N-dimethyl-N-hydroxyethyl ammonium bromide, 3-dimethylamino-2-(cholest-5-en-3-beta-oxybutan-4-oxy)-1-(cis,ci-s-9,12-octadecadienoxy) propane, 2-[5′-(cholest-5-en-3-beta-oxy)-3′-oxapentoxy)-3-dimethyl-1-(cis,cis-9′,12′-octadecadienoxy) propane, N,N-dimethyl-3,4-dioleyloxybenzylamine, 1,2-N,N′-dioleylcarbamyl-3-dimethylaminopropane, 2,3-Dilinoleoyloxy-N,N-dimethylpropylamine, 1,2-N,N′-Dilinoleylcarbamyl-3-dimethylaminopropane, 1,2-Dilinoleoylcarbamyl-3-dimethylaminopropane, 2,2-dilinoleyl-4-dimethylaminomethyl-[1,3]-dioxolane, 2,2-dilinoleyl-4-dimethylaminoethyl-[1,3]-dioxolane, and 2-(2,2-di((9Z,12Z)-octadeca-9,12-dien-1-yl)-1,3-dioxolan-4-yl)-N,N-dimethylethanamine (DLin-KC2-DMA)), or mixtures thereof.

In some embodiments, non-cationic lipids can be used. As used herein, the phrase “non-cationic lipid” refers to any neutral, zwitterionic, or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), DOPE, palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone or can be used in combination with other excipients, for example, cationic lipids.

DNA vectors containing nucleic acids such as those described herein also are also provided. A “DNA vector” is a replicon, such as a plasmid, phage, or cosmid, into which another DNA segment may be inserted so as to bring about the replication of the inserted segment. Generally, a DNA vector is capable of replication when associated with the proper control elements. Suitable vector backbones include, for example, those routinely used in the art such as plasmids, viruses, artificial chromosomes, BACs, YACs, or PACs. The term “DNA vector” includes cloning and expression vectors, as well as viral vectors and integrating vectors. An “expression vector” is a vector that includes a regulatory region. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The DNA vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The DNA vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region (e.g. a promoter) and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Direct injection of adenoviral vectors into lung tumors has been a routine procedure in clinical trials evaluating gene therapy of lung cancer. Dong et al., J. Int. Med. Res. 36:1273-87 (2008); Li et al., Cancer Gene Ther. 20:251-59 (2013); Zhou et al., Cancer Gene Ther. 23:1-6 (2016). Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al., BioTechniques 34:167-71 (2003). A large variety of such vectors are known in the art and are generally available.

Suitable nucleic acid delivery systems include recombinant viral vector, typically sequence from at least one of an adenovirus, adenovirus-associated virus (AAV), helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, in some embodiments about one polynucleotide. In embodiments in which the polynucleotide is to be administered with a non-viral vector, use of between from about 0.1 ng to about 4000 μg will often be useful e.g., about 0.1 ng to about 3900 μg, about 0.1 ng to about 3800 μg, about 0.1 ng to about 3700 μg, about 0.1 ng to about 3600 μg, about 0.1 ng to about 3500 μg, about 0.1 ng to about 3400 μg, about 0.1 ng to about 3300 μg, about 0.1 ng to about 3200 μg, about 0.1 ng to about 3100 μg, about 0.1 ng to about 3000 μg, about 0.1 ng to about 2900 μg, about 0.1 ng to about 2800 μg, about 0.1 ng to about 2700 μg, about 0.1 ng to about 2600 μg, about 0.1 ng to about 2500 μg, about 0.1 ng to about 2400 μg, about 0.1 ng to about 2300 μg, about 0.1 ng to about 2200 μg, about 0.1 ng to about 2100 μg, about 0.1 ng to about 2000 μg, about 0.1 ng to about 1900 μg, about 0.1 ng to about 1800 μg, about 0.1 ng to about 1700 μg, about 0.1 ng to about 1600 μg, about 0.1 ng to about 1500 μg, about 0.1 ng to about 1400 μg, about 0.1 ng to about 1300 μg, about 0.1 ng to about 1200 μg, about 0.1 ng to about 1100 μg, about 0.1 ng to about 1000 μg, about 0.1 ng to about 900 μg, about 0.1 ng to about 800 μg, about 0.1 ng to about 700 μg, about 0.1 ng to about 600 μg, about 0.1 ng to about 500 μg, about 0.1 ng to about 400 μg, about 0.1 ng to about 300 μg, about 0.1 ng to about 200 μg, about 0.1 ng to about 100 μg, about 0.1 ng to about 90 μg, about 0.1 ng to about 80 μg, about 0.1 ng to about 70 μg, about 0.1 ng to about 60 μg, about 0.1 ng to about 50 μg, about 0.1 ng to about 40 μg, about 0.1 ng to about 30 μg, about 0.1 ng to about 20 μg, about 0.1 ng to about 10 μg, about 0.1 ng to about 1 μg, about 0.1 ng to about 900 ng, about 0.1 ng to about 800 ng, about 0.1 ng to about 700 ng, about 0.1 ng to about 600 ng, about 0.1 ng to about 500 ng, about 0.1 ng to about 400 ng, about 0.1 ng to about 300 ng, about 0.1 ng to about 200 ng, about 0.1 ng to about 100 ng, about 0.1 ng to about 90 ng, about 0.1 ng to about 80 ng, about 0.1 ng to about 70 ng, about 0.1 ng to about 60 ng, about 0.1 ng to about 50 ng, about 0.1 ng to about 40 ng, about 0.1 ng to about 30 ng, about 0.1 ng to about 20 ng, about 0.1 ng to about 10 ng, about 0.1 ng to about 1 ng, about 1 ng to about 4000 μg, about 1 ng to about 3900 μg, about 1 ng to about 3800 μg, about 1 ng to about 3700 μg, about 1 ng to about 3600 μg, about 1 ng to about 3500 μg, about 1 ng to about 3400 μg, about 1 ng to about 3300 μg, about 1 ng to about 3200 μg, about 1 ng to about 3100 μg, about 1 ng to about 3000 μg, about 1 ng to about 2900 μg, about 1 ng to about 2800 μg, about 1 ng to about 2700 μg, about 1 ng to about 2600 μg, about 1 ng to about 2500 μg, about 1 ng to about 2400 μg, about 1 ng to about 2300 μg, about 1 ng to about 2200 μg, about 1 ng to about 2100 μg, about 1 ng to about 2000 μg, about 1 ng to about 1900 μg, about 1 ng to about 1800 μg, about 1 ng to about 1700 μg, about 1 ng to about 1600 μg, about 1 ng to about 1500 μg, about 1 ng to about 1400 μg, about 1 ng to about 1300 μg, about 1 ng to about 1200 μg, about 1 ng to about 1100 μg, about 1 ng to about 1000 μg, about 1 ng to about 900 μg, about 1 ng to about 800 μg, about 1 ng to about 700 μg, about 1 ng to about 600 μg, about 1 ng to about 500 μg, about 1 ng to about 400 μg, about 1 ng to about 300 μg, about 1 ng to about 200 μg, about 1 ng to about 100 μg, about 1 ng to about 90 μg, about 1 ng to about 80 μg, about 1 ng to about 70 μg, about 1 ng to about 60 μg, about 1 ng to about 50 μg, about 1 ng to about 40 μg, about 1 ng to about 30 μg, about 1 ng to about 20 μg, about 1 ng to about 10 μg, about 1 ng to about 1 μg, about 1 ng to about 900 ng, about 1 ng to about 800 ng, about 1 ng to about 700 ng, about 1 ng to about 600 ng, about 1 ng to about 500 ng, about 1 ng to about 400 ng, about 1 ng to about 300 ng, about 1 ng to about 200 ng, about 1 ng to about 100 ng, about 1 ng to about 90 ng, about 1 ng to about 80 ng, about 1 ng to about 70 ng, about 1 ng to about 60 ng, about 1 ng to about 50 ng, about 1 ng to about 40 ng, about 1 ng to about 30 ng, about 1 ng to about 20 ng, about 1 ng to about 10 ng, about 10 ng to about 4000 μg, about 20 ng to about 4000 μg, about 30 ng to about 4000 μg, about 40 ng to about 4000 μg, about 50 ng to about 4000 μg, about 60 ng to about 4000 μg, about 70 ng to about 4000 μg, about 80 ng to about 4000 μg, about 90 ng to about 4000 μg, about 100 ng to about 4000 μg, about 200 ng to about 4000 μg, about 300 ng to about 4000 μg, about 400 ng to about 4000 μg, about 500 ng to about 4000 μg, about 600 ng to about 4000 μg, about 700 ng to about 4000 μg, about 800 ng to about 4000 μg, about 900 ng to about 4000 μg, about 1 μg to about 4000 μg, 10 μg to about 4000 μg, 20 μg to about 4000 μg, 30 μg to about 4000 μg, 40 μg to about 4000 μg, 50 μg to about 4000 μg, 60 μg to about 4000 μg, 70 μg to about 4000 μg, 80 μg to about 4000 μg, 90 μg to about 4000 μg, 100 μg to about 4000 μg, 200 μg to about 4000 μg, 300 μg to about 4000 μg, 400 μg to about 4000 μg, 500 μg to about 4000 μg, 600 μg to about 4000 μg, 700 μg to about 4000 μg, 800 μg to about 4000 μg, 900 μg to about 4000 μg, 1000 μg to about 4000 μg, 1100 μg to about 4000 μg, 1200 μg to about 4000 μg, 1300 μg to about 4000 μg, 1400 μg to about 4000 μg, 1500 μg to about 4000 μg, 1600 μg to about 4000 μg, 1700 μg to about 4000 μg, 1800 μg to about 4000 μg, 1900 μg to about 4000 μg, 2000 μg to about 4000 μg, 2100 μg to about 4000 μg, 2200 μg to about 4000 μg, 2300 μg to about 4000 μg, 2400 μg to about 4000 μg, 2500 μg to about 4000 μg, 2600 μg to about 4000 μg, 2700 μg to about 4000 μg, 2800 μg to about 4000 μg, 2900 μg to about 4000 μg, 3000 μg to about 4000 μg, 3100 μg to about 4000 μg, 3200 μg to about 4000 μg, 3300 μg to about 4000 μg, 3400 μg to about 4000 μg, 3500 μg to about 4000 μg, 3600 μg to about 4000 μg, 3700 μg to about 4000 μg, 3800 μg to about 4000 μg, or 3900 μg to about 4000 μg.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector (see, e.g., Geller et al., J. Neurochem. 64:487-96 (1995); Lim et al., in DNA Cloning: Mammalian Systems, D. Glover, Ed. (Oxford Univ. Press, Oxford England) (1995); Geller et al., Proc. Natl. Acad. Sci. USA 90:7603-07 (1993); Geller et al., Proc. Natl. Acad. Sci. USA 87:1149-53 (1990)), Adenovirus Vectors (see, e.g., Le Gal LaSalle et al., Science 259:988-90 (1993); Davidson et al., Nat. Genet. 3:219-23 (1993); Yang et al., J. Virol. 69:2004-15 (1995)), and Adeno-associated Virus Vectors (see, e.g., Kaplitt et al., Nat. Genet. 8:148-54 (1994)).

If desired, the polynucleotides described here may also be used with a microdelivery vehicle such as cationic liposomes, adenoviral vectors, and exosomes. For a review of the procedures for liposome preparation, targeting and delivery of contents, see Mannino et al., BioTechniques 6:682-90 (1988). See also Feigner et al., Bethesda Res. Lab. Focus 11 (2): 21 (1989) and Maurer, Bethesda Res. Lab. Focus 11 (2): 25 (1989). In some embodiments, exosomes may be used for delivery of a nucleic acid encoding a CRISPR endonuclease and/or guide RNA to a target cell, e.g. a cancer cell. Exosomes are nanosized vesicles secreted by a variety of cells and are comprised of cellular membranes. Exosomes can attach to target cells by a range of surface adhesion proteins and vector ligands (tetraspanins, integrins, CD11b and CD18 receptors), and deliver their payload to target cells. Several studies indicate that exosomes have a specific cell tropism, according to their characteristics and origin, which can be used to target them to disease tissues and/or organs. See Batrakova et al., J. Control. Release 219:396-405 (2015). For example, cancer-derived exosomes function as natural carriers that can efficiently deliver CRISPR/Cas9 plasmids to cancer cells. See Kim et al., J. Control. Release 266:8-16 (2017).

Replication-defective recombinant adenoviral vectors, can be produced in accordance with known techniques. See Quantin et al., Proc. Natl. Acad. Sci. USA 89:2581-84 (1992); Stratford-Perricadet et al., J. Clin. Invest. 90:626-30 (1992); Rosenfeld et al., Cell 68:143-55 (1992).

Another delivery method is to use single stranded DNA producing vectors which can produce the expressed products intracellularly. See, e.g., Chen et al., BioTechniques, 34:167-71 (2003).

In some embodiments, the methods, compositions, and combinations disclosed herein can be used for the treatment of tumors. In other embodiments, treatment of a tumor includes inhibiting tumor growth, promoting tumor reduction, or both inhibiting tumor growth and promoting tumor reduction.

In some embodiments, intratumoral or peritumoral delivery of the presently described CRISPR/Cas systems can result in, e.g., about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more reduction in tumor size as compared to an untreated tumor.

In some embodiments, intratumoral or peritumoral delivery of the presently described CRISPR/Cas systems can result in, e.g., about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more inhibition in tumor growth as compared to an untreated tumor.

In certain embodiments, the tumor is treated with an additional agent, e.g. a chemotherapeutic agent. In certain embodiments, treatment with the chemotherapeutic agent is initiated at the same time as treatment with the therapeutically effective amount of a CRISPR/Cas system. In certain embodiments, the treatment with the chemotherapeutic agent is initiated after the treatment with the therapeutically effective amount of a CRISPR/Cas system is initiated. In certain embodiments, treatment with the chemotherapeutic agent is initiated at before the treatment with the therapeutically effective amount of a CRISPR/Cas system.

In certain embodiments, the therapeutically effective amount of a CRISPR/Cas system of the present disclosure may be utilized for the treatment of a tumor wherein the subject has failed at least one prior chemotherapeutic regimen. For example, in some embodiments, the tumor is resistant to one or more chemotherapeutic agents. Accordingly, the present disclosure provides methods of treating a tumor in a subject, wherein the subject has failed at least one prior chemotherapeutic regimen for the cancer, comprising administering the therapeutically effective amount of a CRISPR/Cas systems as described herein to the subject in an amount sufficient to treat the tumor. The therapeutically effective amount of a CRISPR/Cas systems described herein may also be utilized for inhibiting tumor cell growth in a subject wherein the subject has failed at least one prior chemotherapeutic regimen. Accordingly, the present disclosure further provides methods of inhibiting tumor cell growth in a subject, e.g, wherein the subject has failed at least one prior chemotherapeutic regimen, comprising administering the pharmaceutical compositions described herein to the subject, such that tumor cell growth is inhibited.

Small molecule chemotherapeutic agents generally belong to various classes including, for example: 1. Topoisomerase II inhibitors (cytotoxic antibiotics), such as the anthracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; 2. Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; 3. Alkylating agents, such as nitrogen mustards, ethyleneimine compounds, alkyl sulphonates and other compounds with an alkylating action such as nitrosoureas, dacarbazine, cyclophosphamide, ifosfamide and melphalan; 4. Antimetabolites (nucleoside inhibitors), for example, folates, e.g., folic acid, fiuropyrimidines, purine or pyrimidine analogues such as 5-fluorouracil, capecitabine, gemcitabine, methotrexate, and edatrexate; 5. Topoisomerase I inhibitors, such as topotecan, irinotecan, and 9-nitrocamptothecin, camptothecin derivatives, and retinoic acid; and 6. Platinum compounds/complexes, such as cisplatin, oxaliplatin, and carboplatin. Exemplary chemotherapeutic agents for use in the methods of disclosed herein include, but are not limited to, amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), capecitabine, ftorafur, 5′deoxyflurouridine, UFT, eniluracil, deoxycytidine, 5-azacytosine, 5-azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973 (and analogs thereof), JM-216 (and analogs thereof), epirubicin, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, acyclovir, valacyclovir, ganciclovir, amantadine, rimantadine, lamivudine, zidovudine, bevacizumab, trastuzumab, rituximab, Pentostatin, floxuridine, fludarabine, hydroxyurea, ifosfamide, idarubicin, mesna, irinotecan, mitoxantrone, topotecan, leuprolide, megestrol, melphalan, plicamycin, mitotane, pegaspargase, pipobroman, tamoxifen, teniposide, testolactone, thiotepa, uracil mustard, vinorelbine, chlorambucil, mTor, epidermal growth factor receptor (EGFR), and fibroblast growth factors (FGF) and combinations thereof which are readily apparent to one of skill in the art based on the appropriate standard of care for a particular tumor or cancer. In a particular embodiment, the chemotherapeutic agent is selected from the group consisting of cisplatin, vinorelbine, carboplatin, and combinations thereof (e.g., cisplatin and vinorelbine; cisplatin and carboplatin; vinorelbine and carboplatin; cisplatin, vinorelbine, and carboplatin).

In some embodiments, intratumoral or peritumoral delivery of the presently described CRISPR/Cas systems can result in, e.g., about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more reduction in the amount of chemotherapeutic agent used in the treatment of a subject.

### Cancer Genes

In some embodiments, the cancer gene is a cancer driver gene, a disease-causing gene, oncogene, or variant tumor suppressor such as, e.g., NRF2, EGFR, EIF1AX, GNA11, SF3B1, BAP1, PBRM1, ATM, SETD2, KDM6A, CUL3, MET, SMARCA4, U2AF1, RBM10, STK11, NF1, NF2, IDH1, IDH2, PTPN11, MAX, TCF12, HIST1H1E, LZTR1, KIT, RAC1, ARID2, BRD4, BRD7, BARF1, NRAS, RNF43, SMAD4, ARID1A, ARID1B, KRAS, APC, SMAD2, SMAD3, ACVR2A, GNAS, HRAS, STAG2, FGFR3, FGFR4, RHOA, CDKN1A, ERBB3, KANSL1, RB1, TP53, CDKN2A, CDKN2B, CDKN2C, KEAP1, CASP8, TGFBR2, HLA-B, MAPK1, NOTCH1, NOTCH2, NOTCH3, HLA-A, RASA1, EPHA2, EPHA3, EPHA5, EPHA7, NSD1, ZNF217, ZNF750, KLF5, EP300, FAT1, PTEN, FBXW7, PIK3CA, PIK3CB, PIK3C2B, PIK3CG, RUNX1, RUNX1T1, DNMT3A, SMC1A, ERBB2, AKT1, AKT2, AKT3, MAP3K1, FOXA1, BRCA1, BRCA2, CDH1, PIK3R1, PPP2R1A, BCOR, BCORL1, ARHGAP35, FGFR2, CHD4, CTCF, CTNNA1, CTNNB1, SPOP, TMSB4X, PIM1, CD70, CD79A, CD79B, B2M, CARD11, MYD88, BTG1, BTG2, TNFAIP3, MEN1, PRKAR1A, PDGFRA, PDGFRB, SPTA1, GABRA6, KEL, SMARCB1, ZBTB7B, BCL2, BCL2L1, BCL2L2, BCL2L11, RFC1, MAP3K4, CSDE1, EPAS1, RET, LATS2, EEF2, CYLD, HUWE1, MYH9, AJUBA, FLNA, ERBB4, CNBD1, DMD, MUC6, FAM46C, FAM46D, PLCG1, PLCG2, NIPBL, FUBP1, CIC, ZBTB2, ZBTB20, ZCCHC12, TGIF1, SOX2, SOX9, SOX10, PCBP1, ZFP36L2, TCF7L2, AMER1, KDM5A, KDM5C, MTOR, VHL, KIF1A, TCEB1, TXNIP, CUL1, TSC1, ELF3, RHOB, PSIP1, SF1, FOXQ1, GNA13, DIAPH2, ZFP36L1, ERCC2, SPTAN1, RXRA, ASXL2, CREBBP, CREB3L3, ALB, DHX9, XPO1, RPS6KA3, IL6ST, TSC2, EEF1A1, WHSC1, APOB, NUP133, AXIN1, PHF6, TET2, WT1, FLT3, FLT4, SMC3, CEBPA, RAD21, RAD50, RAD51, PTPDC1, ASXL1, EZH2, NPM1, SRSF2, GNAQ, PLCB4, CYSLTR2, CDKN1B, CBFB, NCOR1, PTPRD, TBX3, GPS2, GATA1, GATA2, GATA3, GATA4, GATA6, MAP2K4, PTCH1, PTMA, LATS1, POLRMT, CDK4, COL5A1, PPP6C, MECOM, DACH1, MAP2K1, MAP2K2, RQCD1, DDX3X, NUP93, PPM1D, CHD2, CHD3, CCND1, CCND2, CCND3, ACVR1, KMT2A, KMT2B, KMT2C, KMT2D, SIN3A, SCAF4, DICER1, FOXA2, CTNND1, MYC, MYCL, MYCN, SOX17, ARID5B, ATR, INPPL1, INPP4B, ATF7IP, ZMYM2, ZFHX3, PDS5B, SOS1, TAF1, PIK3R2, RPL22, RRAS2, MSH2, MSH6, CKD12, ZNF133, ZNF703, MED12, ZMYM3, GTF21, RIT1, MGA, ABL1, BRAF, CHEK1, FANCC, JAK2, MITF, PDCD1LG2, STAT4, ABL2, CHEK2, FANCD2, JAK3, MLH1, FANCE, JUN, MPL, RICTOR, SUFU, FANCF, GID4, KAT6A, MRE11A, PDK1, SYK, BRIP1, CRKL, FANCG, GLI1, CRLF2, FANCL, RPTOR, ALK, BTK, CSF1R, FAS, TERC, C11orf30, KDR, MUTYH, SDHA, AR, FGF10, GPR124, SDHB, ARAF, CBL, FGF14, GRIN2A, SDHC, ARFRP1, FGF19, GRM3, KLHL6, PMS2, SDHD, TNFRSF14, DAXX, FGF23, GSK3B, POLD1, TOP1, DDR2, FGF3, H3F3A, POLE, TOP2A, CCNE1, FGF4, HGF, SLIT2, CD274, FGF6, HNF1A, NFKBIA, PRDM1, DOT1L, FGFR1, LMO1, NKX2-1, PREX2, HSD3B1, LRP1B, TSHR, ATRX, CDC73, HSP90AA1, PRKCI, AURKA, PRKDC, VEGFA, AURKB, CDK12, FH, MAGI2, PRSS8, SMO, FLCN, IGF1R, SNCAIP, WISP3, AXL, CDK6, EPHB1, FLT1, IGF2, SOCS1, CDK8, IKBKE, NTRK1, BARD1, IKZF1, NTRK2, QKI, FOXL2, IL7R, MCL1, NTRK3, ERG, FOXP1, INHBA, MDM2, SPEN, ERRFI1, FRS2, MDM4, PAK3, BCL6, ESR1, IRF2, PALB2, RAF1, IRF4, MEF2B, PARK2, RANBP2, SRC, IRS2, PAX5, RARA, BLM, FANCA, JAK1, FCRL4, LIG4, MAR, PWWP3A, MUC16, MUC17, FCGBP, FAT17, MMSET, IRTA2, TTN, DST, or STAT3.

In some embodiments, the cancer gene is Nuclear Factor Erythroid 2-Related Factor (NRF2, NFE2L2). NRF2 is considered the master regulator of 100-200 target genes involved in cellular responses to oxidative/electrophilic stress. Targets include glutathione (GSH) mediators, antioxidants and genes controlling efflux pumps. (Hayden et al., Urol. Oncol. Semin. Orig. Investig. 32:806-14 (2014)). NRF2 is also known to regulate expression of genes involved in protein degradation and detoxification and is negatively regulated by Kelch-like ECH-associated protein 1 (KEAP1), a substrate adapter for the Cul3-dependent E3 ubiquitin ligase complex. Under normal conditions, Keap1 constantly targets NRF2 for ubiquitin-dependent degradation maintaining low expression of NRF2 on downstream target genes. However, chemotherapy has been shown to activate transcriptional activity of the NRF2 target genes often triggering a cytoprotective response; enhanced expression of NRF2 occurs in response to environmental stress or detrimental growth conditions. Other mechanisms that lead to NRF2 upregulation include mutations in KEAP1 or epigenetic changes of the promoter region. The upregulation of NRF2 expression leads to an enhanced resistance of cancer cells to chemotherapeutic drugs, which by their very action induce an unfavorable environment for cell proliferation. Indeed, Hayden et al. (ibid) have clearly demonstrated that increased NRF2 expression leads to the resistance of cancer cells to chemotherapeutic drugs including cisplatin. Singh et al. (2010, Antioxidants & Redox Signaling 13) also showed that constitutive expression of NRF2 leads to radioresistance, and inhibition of NRF2 causes increased endogenous reactive oxygen species (ROS) levels as well as decreased survival. Torrente et al. (Oncogene (2017). doi: 10.1038/onc.2017.221) identified crosstalk between NRF2 and the homeodomain interacting protein kinase two, HIPK2, demonstrating that HIPK2 exhibits a cytoprotective effect through NRF2.

By using CRISPR/Cas9, it is possible to target and knock out the mutated NRF2 protein causing chemoresistance, while not disrupting the function of wildtype NRF2 protein (PCT/US2020/034369, incorporated herein by reference in its entirety). Thus, some embodiments are directed to reducing or, in some embodiments, eliminating expression of variant NRF2s found only in cancer cells and not in non-cancerous cells. These variants are commonly found within the Neh2 Domain of NRF2, which is known as the KEAP1 binding domain. In some embodiments, the NRF2 mutations can be those found in Table 1 below.

TABLE 1

| | | | Notes/Amino |
|---|---|---|---|
| | | | Acid substitution |
| SEQ | | | (relative to |
| ID | Sequence, with substitution | Base position in | position in SEQ |
| NO: | identified in brackets | SEQ ID NO: 15 | ID NO: 8) |
| 1 | TTTGATTGACATACTTTGGAGGC(C > G) | 205-227 | Q26E |
| 2 | CTTACTCCAAGATCTATATCTT(T > G)G | 249-227 | Reverse |
| | | | complement; |
| | | | Q26P |
| 3 | ATACTTTGGAGGCAAGATATAGA(A > G) | 215-237 | D29G |
| 4 | AGGCAAGATATAGATCTTGGAGT(T > G) | 224-246 | V32G |
| 5 | GATATAGATCTTGGAGTAAGTC(C > G)G | 230-252 | R34G |
| 6 | TGACTGAAGTCAAATACTTCTC(C > G)G | 273-251 | Reverse |
| | | | complement; |
| | | | R34P |
| 7 | TTCATCTAGTTGTAACTGAGCGA(A > G) | 385-363 | Reverse |
| | | | complement; |
| | | | F71S |
| 8 | ATTCACCTGTCTCTTCATCTAGT(T > G) | 398-376 | Reverse |
| | | | complement; |
| | | | Q75H |
| 9 | CTCAGTTACAACTAGATGAAGA(A > G)G | 366-388 | E79G |
| 10 | TGAATTGGGAGAAATTCACCTGT(T > G) | 411-389 | Reverse |
| | | | complement; |
| | | | T80P |
| 11 | CAACTAGATGAAGAGACAGGTGA(A > G) | 374-396 | E82W |
| 12 | ATAATAGCTCCTCCCAAACTTGC(C > G) | 728-706 | Reverse |
| | | | complement; |
| | | | E185D |
| 13 | TTTTTCGCTCAGTTACAACTAGA(A > G) | 360-381 | D77G |
| 14 | CAACTAGATGAAGAGACAGGTGA(A > G) | 374-396 | E82G |

PAM sequence underlined.

| NRF2 SEQ ID NO: 15: | |
|---|---|
| 1 | gattaccgag tgccggggag cccggaggag ccgccgacgc agccgccacc gccgccgccg |
| 61 | ccgccaccag agccgccctg tccgcgccgc gcctcggcag ccggaacagg gccgccgtcg |
| 121 | gggagcccca acacacggtc cacagctcat catgatggac ttggagctgc cgccgccggg |
| 181 | actcccgtcc cagcaggaca tggatttgat tgacatactt tggaggcaag atatagatct |
| 241 | tggagtaagt cgagaagtat ttgacttcag tcagcgacgg aaagagtatg agctggaaaa |
| 301 | acagaaaaaa cttgaaaagg aaagacaaga acaactccaa aaggagcaag agaaagcctt |
| 361 | tttcgctcag ttacaactag atgaagagac aggtgaattt ctcccaattc agccagccca |
| 421 | gcacatccag tcagaaacca gtggatctgc caactactcc caggttgccc acattcccaa |
| 481 | atcagatgct ttgtactttg atgactgcat gcagcttttg gcgcagacat tcccgtttgt |
| 541 | agatgacaat gaggtttctt cggctacgtt tcagtcactt gttcctgata ttcccggtca |
| 601 | catcgagagc ccagtcttca ttgctactaa tcaggctcag tcacctgaaa cttctgttgc |
| 661 | tcaggtagcc cctgttgatt tagacggtat gcaacaggac attgagcaag tttgggagga |
| 721 | gctattatcc attcctgagt tacagtgtct taatattgaa aatgacaagc tggttgagac |
| 781 | taccatggtt ccaagtccag aagccaaact gacagaagtt gacaattatc atttttactc |
| 841 | atctataccc tcaatggaaa aagaagtagg taactgtagt ccacattttc ttaatgcttt |
| 901 | tgaggattcc ttcagcagca tcctctccac agaagacccc aaccagttga cagtgaactc |
| 961 | attaaattca gatgccacag tcaacacaga ttttggtgat gaattttatt ctgctttcat |
| 1021 | agctgagccc agtatcagca acagcatgcc ctcacctgct actttaagcc attcactctc |
| 1081 | tgaacttcta aatgggccca ttgatgtttc tgatctatca ctttgcaaag ctttcaacca |
| 1141 | aaaccaccct gaaagcacag cagaattcaa tgattctgac tccggcattt cactaaacac |
| 1201 | aagtcccagt gtggcatcac cagaacactc agtggaatct tccagctatg gagacacact |
| 1261 | acttggcctc agtgattctg aagtggaaga gctagatagt gcccctggaa gtgtcaaaca |
| 1321 | gaatggtcct aaaacaccag tacattcttc tggggatatg gtacaaccct tgtcaccatc |
| 1381 | tcaggggcag agcactcacg tgcatgatgc ccaatgtgag aacacaccag agaaagaatt |
| 1441 | gcctgtaagt cctggtcatc ggaaaacccc attcacaaaa gacaaacatt caagccgctt |
| 1501 | ggaggctcat ctcacaagag atgaacttag ggcaaaagct ctccatatcc cattccctgt |
| 1561 | agaaaaaatc attaacctcc ctgttgttga cttcaacgaa atgatgtcca aagagcagtt |
| 1621 | caatgaagct caacttgcat taattcggga tatacgtagg aggggtaaga ataaagtggc |
| 1681 | tgctcagaat tgcagaaaaa gaaaactgga aaatatagta gaactagagc aagatttaga |
| 1741 | tcatttgaaa gatgaaaaag aaaaattgct caaagaaaaa ggagaaaatg acaaaagcct |
| 1801 | tcacctactg aaaaaacaac tcagcacctt atatctcgaa gttttcagca tgctacgtga |
| 1861 | tgaagatgga aaaccttatt ctcctagtga atactccctg cagcaaacaa gagatggcaa |
| 1921 | tgttttcctt gttcccaaaa gtaagaagcc agatgttaag aaaaactaga tttaggagga |
| 1981 | tttgaccttt tctgagctag tttttttgta ctattatact aaaagctcct actgtgatgt |
| 2041 | gaaatgctca tactttataa gtaattctat gcaaaatcat agccaaaact agtatagaaa |
| 2101 | ataatacgaa actttaaaaa gcattggagt gtcagtatgt tgaatcagta gtttcacttt |
| 2161 | aactgtaaac aatttcttag gacaccattt gggctagttt ctgtgtaagt gtaaatacta |
| 2221 | caaaaactta tttatactgt tcttatgtca tttgttatat tcatagattt atatgatgat |
| 2281 | atgacatctg gctaaaaaga aattattgca aaactaacca ctatgtactt ttttataaat |
| 2341 | actgtatgga caaaaaatgg cattttttat attaaattgt ttagctctgg caaaaaaaaa |
| 2401 | aaattttaag agctggtact aataaaggat tattatgact gttaaa |

| SEQ ID NO: 16 | |
|---|---|
| 1 | agacgtccgg gcagcccccg gcgcagcgcg gccgcagcag cctccgcccc ccgcacggtg |
| 61 | tgagcgcccg acgcggccga ggcggccgga gtcccgagct agccccggcg gccgccgccg |
| 121 | cccagaccgg acgacaggcc acctcgtcgg cgtccgcccg agtccccgcc tcgccgccaa |
| 181 | cgccacaacc accgcgcacg gccccctgac tccgtccagt attgatcggg agagccggag |
| 241 | cgagctcttc ggggagcagc gatgcgaccc tccgggacgg ccggggcagc gctcctggcg |
| 301 | ctgctggctg cgctctgccc ggcgagtcgg gctctggagg aaaagaaagt ttgccaaggc |
| 361 | acgagtaaca agctcacgca gttgggcact tttgaagatc attttctcag cctccagagg |
| 421 | atgttcaata actgtgaggt ggtccttggg aatttggaaa ttacctatgt gcagaggaat |
| 481 | tatgatcttt ccttcttaaa gaccatccag gaggtggctg gttatgtcct cattgccctc |
| 541 | aacacagtgg agcgaattcc tttggaaaac ctgcagatca tcagaggaaa tatgtactac |
| 601 | gaaaattcct atgccttagc agtcttatct aactatgatg caaataaaac cggactgaag |
| 661 | gagctgccca tgagaaattt acaggaaatc ctgcatggcg ccgtgcggtt cagcaacaac |
| 721 | cctgccctgt gcaacgtgga gagcatccag tggcgggaca tagtcagcag tgactttctc |
| 781 | agcaacatgt cgatggactt ccagaaccac ctgggcagct gccaaaagty tgatccaagc |
| 841 | tgtcccaatg ggagctgctg gggtgcagga gaggagaact gccagaaact gaccaaaatc |
| 901 | atctgtgccc agcagtgctc cgggcgctgc cgtggcaagt cccccagtga ctgctgccac |
| 961 | aaccagtgtg ctgcaggctg cacaggcccc cgggagagcg actgcctggt ctgccgcaaa |
| 1021 | ttccgagacg aagccacgtg caaggacacc tgccccccac tcatgctcta caaccccacc |
| 1081 | acgtaccaga tggatgtgaa ccccgagggc aaatacagct ttggtgccac ctgcgtgaag |
| 1141 | aagtgtcccc gtaattatgt ggtgacagat cacggctcgt gcgtccgagc ctgtggggcc |
| 1201 | gacagctatg agatggagga agacggcgtc cgcaagtgta agaagtgcga agggccttgc |
| 1261 | cgcaaagtgt gtaacggaat aggtattggt gaatttaaag actcactctc cataaatgct |
| 1321 | acgaatatta aacacttcaa aaactgcacc tccatcagtg gcgatctcca catcctgccg |
| 1381 | gtggcattta ggggtgactc cttcacacat actcctcctc tggatccaca ggaactggat |
| 1441 | attctgaaaa ccgtaaagga aatcacaggg tttttgctga ttcaggcttg gcctgaaaac |
| 1501 | aggacggacc tccatgcctt tgagaaccta gaaatcatac gcggcaggac caagcaacat |
| 1561 | ggtcagtttt ctcttgcagt cgtcagcctg aacataacat ccttgggatt acgctccctc |
| 1621 | aaggagataa gtgatggaga tgtgataatt tcaggaaaca aaaatttgtg ctatgcaaat |
| 1681 | acaataaact ggaaaaaact gtttgggacc tccggtcaga aaaccaaaat tataagcaac |
| 1741 | agaggtgaaa acagctgcaa ggccacaggc caggtctgcc atgccttgtg ctcccccgag |
| 1801 | ggctgctggg gcccggagcc cagggactgc gtctcttgcc ggaatgtcag ccgaggcagg |
| 1861 | gaatgcgtgg acaagtgcaa ccttctggag ggtgagccaa gggagtttgt ggagaactct |
| 1921 | gagtgcatac agtgccaccc agagtgcctg cctcaggcca tgaacatcac ctgcacagga |
| 1981 | cggggaccag acaactgtat ccagtgtgcc cactacattg acggccccca ctgcgtcaag |
| 2041 | acctgcccgg caggagtcat gggagaaaac aacaccctgg tctggaagta cgcagacgcc |
| 2101 | ggccatgtgt gccacctgtg ccatccaaac tgcacctacg gatgcactgg gccaggtctt |
| 2161 | gaaggctgtc caacgaatgg gcctaagatc ccgtccatcg ccactgggat ggtgggggcc |
| 2221 | ctcctcttgc tgctggtggt ggccctgggg atcggcctct tcatgcgaag gcgccacatc |
| 2281 | gttcggaagc gcacgctgcg gaggctgctg caggagaggg agcttgtgga gcctcttaca |
| 2341 | cccagtggag aagctcccaa ccaagctctc ttgaggatct tgaaggaaac tgaattcaaa |
| 2401 | aagatcaaag tgctgggctc cggtgcgttc ggcacggtgt ataagggact ctggatccca |
| 2461 | gaaggtgaga aagttaaaat tcccgtcgct atcaaggaat taagagaagc aacatctccg |
| 2521 | aaagccaaca aggaaatcct cgatgaagcc tacgtgatgg ccagcgtgga caacccccac |
| 2581 | gtgtgccgcc tgctgggcat ctgcctcacc tccaccgtgc agctcatcac gcagctcatg |
| 2641 | cccttcggct gcctcctgga ctatgtccgg gaacacaaag acaatattgg ctcccagtac |
| 2701 | ctgctcaact ggtgtgtgca gatcgcaaag ggcatgaact acttggagga ccgtcgcttg |
| 2761 | gtgcaccgcg acctggcagc caggaacgta ctggtgaaaa caccgcagca tgtcaagatc |
| 2821 | acagattttg ggctggccaa actgctgggt gcggaagaga aagaatacca tgcagaagga |
| 2881 | ggcaaagtgc ctatcaagtg gatggcattg gaatcaattt tacacagaat ctatacccac |
| 2941 | cagagtgatg tctggagcta cggggtgact gtttgggagt tgatgacctt tggatccaag |
| 3001 | ccatatgacg gaatccctgc cagcgagatc tcctccatcc tggagaaagg agaacgcctc |
| 3061 | cctcagccac ccatatgtac catcgatgtc tacatgatca tggtcaagtg ctggatgata |
| 3121 | gacgcagata gtcgcccaaa gttccgtgag ttgatcatcg aattctccaa aatggcccga |
| 3181 | gacccccagc gctaccttgt cattcagggg gatgaaagaa tgcatttgcc aagtcctaca |
| 3241 | gactccaact tctaccgtgc cctgatggat gaagaagaca tggacgacgt ggtggatgcc |
| 3301 | gacgagtacc tcatcccaca gcagggcttc ttcagcagcc cctccacgtc acggactccc |
| 3361 | ctcctgagct ctctgagtgc aaccagcaac aattccaccg tggcttgcat tgatagaaat |
| 3421 | gggctgcaaa gctgtcccat caaggaagac agcttcttgc agcgatacag ctcagacccc |
| 3481 | acaggcgcct tgactgagga cagcatagac gacaccttcc tcccagtgcc tgaatacata |
| 3541 | aaccagtccg ttcccaaaag gcccgctggc tctgtgcaga atcctgtcta tcacaatcag |
| 3601 | cctctgaacc ccgcgcccag cagagaccca cactaccagg acccccacag cactgcagtg |
| 3661 | ggcaaccccg agtatctcaa cactgtccag cccacctgtg tcaacagcac attcgacagc |
| 3721 | cctgcccact gggcccagaa aggcagccac caaattagcc tggacaaccc tgactaccag |
| 3781 | caggacttct ttcccaagga agccaagcca aatggcatct ttaagggctc cacagctgaa |
| 3841 | aatgcagaat acctaagggt cgcgccacaa agcagtgaat ttattggagc atgaccacgg |
| 3901 | aggatagtat gagccctaaa aatccagact ctttcgatac ccaggaccaa gccacagcag |
| 3961 | gtcctccatc ccaacagcca tgcccgcatt agctcttaga cccacagact ggttttgcaa |
| 4021 | cgtttacacc gactagccag gaagtactic cacctcgggc acattttggg aagttgcatt |
| 4081 | cctttgtctt caaactgtga agcatttaca gaaacgcatc cagcaagaat attgtccctt |
| 4141 | tgagcagaaa tttatctttc aaagaggtat atttgaaaaa aaaaaaaagt atatgtgagg |
| 4201 | atttttattg attggggatc ttggagtttt tcattgtcgc tattgatttt tacttcaatg |
| 4261 | ggctcttcca acaaggaaga agcttgctgg tagcacttgc taccctgagt tcatccaggc |
| 4321 | ccaactgtga gcaaggagca caagccacaa gtcttccaga ggatgcttga ttccagtggt |
| 4381 | tctgcttcaa ggcttccact gcaaaacact aaagatccaa gaaggccttc atggccccag |
| 4441 | caggccggat cggtactgta tcaagtcatg gcaggtacag taggataagc cactctgtcc |
| 4501 | cttcctgggc aaagaagaaa cggaggggat ggaattcttc cttagactta cttttgtaaa |
| 4561 | aatgtcccca cggtacttac tccccactga tggaccagtg gtttccagtc atgagcgtta |
| 4621 | gactgacttg tttgtcttcc attccattgt tttgaaactc agtatgctgc ccctgtcttg |
| 4681 | ctgtcatgaa atcagcaaga gaggatgaca catcaaataa taactcggat tccagcccac |
| 4741 | attggattca tcagcatttg gaccaatagc ccacagctga gaatgtggaa tacctaagga |
| 4801 | tagcaccgct tttgttctcg caaaaacgta tctcctaatt tgaggctcag atgaaatgca |
| 4861 | tcaggtcctt tggggcatag atcagaagac tacaaaaatg aagctgctct gaaatctcct |
| 4921 | ttagccatca ccccaacccc ccaaaattag tttgtgttac ttatggaaga tagttttctc |
| 4981 | cttttacttc acttcaaaag ctttttactc aaagagtata tgttccctcc aggtcagctg |
| 5041 | cccccaaacc ccctccttac gctttgtcac acaaaaagtg tctctgcctt gagtcatcta |
| 5101 | ttcaagcact tacagctctg gccacaacag ggcattttac aggtgcgaat gacagtagca |
| 5161 | ttatgagtag tgtggaattc aggtagtaaa tatgaaacta gggtttgaaa ttgataatgc |
| 5221 | tttcacaaca tttgcagatg ttttagaagg aaaaaagttc cttcctaaaa taatttctct |
| 5281 | acaattggaa gattggaaga ttcagctagt taggagccca ccttttttcc taatctgtgt |
| 5341 | gtgccctgta acctgactgg ttaacagcag tcctttgtaa acagtgtttt aaactctcct |
| 5401 | agtcaatatc caccccatcc aatttatcaa ggaagaaatg gttcagaaaa tattttcagc |
| 5461 | ctacagttat gttcagtcac acacacatac aaaatgttcc ttttgctttt aaagtaattt |
| 5521 | ttgactccca gatcagtcag agcccctaca gcattgttaa gaaagtattt gatttttgtc |
| 5581 | tcaatgaaaa taaaactata ttcatttcca ctctattatg ctctcaaata cccctaagca |
| 5641 | tctatactag cctggtatgg gtatgaaaga tacaaagata aataaaacat agtccctgat |
| 5701 | tctaagaaat tcacaattta gcaaaggaaa tggactcata gatgctaacc ttaaaacaac |
| 5761 | gtgacaaatg ccagacagga cccatcagcc aggcactgtg agagcacaga gcagggaggt |
| 5821 | tgggtcctgc ctgaggagac ctggaaggga ggcctcacag gaggatgacc aggtctcagt |
| 5881 | cagcggggag gtggaaagtg caggtgcatc aggggcaccc tgaccgagga aacagctgcc |
| 5941 | agaggcctcc actgctaaag tccacataag gctgaggtca gtcaccctaa acaacctgct |
| 6001 | ccctctaagc caggggatga gcttggagca tcccacaagt tccctaaaag ttgcagcccc |
| 6061 | cagggggatt ttgagctatc atctctgcac atgcttagtg agaagactac acaacatttc |
| 6121 | taagaatctg agattttata ttgtcagtta accactttca ttattcattc acctcaggac |
| 6181 | atgcagaaat atttcagtca gaactgggaa acagaaggac ctacattctg ctgtcactta |
| 6241 | tgtgtcaaga agcagatgat cgatgaggca ggtcagttgt aagtgagtca cattgtagca |
| 6301 | ttaaattcta gtatttttgt agtttgaaac agtaacttaa taaaagagca aaagctattc |
| 6361 | tagctttctt cttcatattt taattttcca ccataaagtt tagttgctaa attctattaa |
| 6421 | ttttaagatt gtgcttccca aaatagttct cacttcatct gtccagggag gcacagttct |
| 6481 | gtctggtaga agccgcaaag cccttagcct cttcacggat ctggcgactg tgatgggcag |
| 6541 | gtcaggagag gagctgccca aagtcccatg attttcacct aacagccctg atcagtcagt |
| 6601 | actcaaagct tggactccat ccctgaaggt cttcctgatt gatagcctgg ccttaatacc |
| 6661 | ctacagaaag cctgtccatt ggctgtttct tcctcagtca gttcctggaa gaccttaccc |
| 6721 | catgacccca gcttcagatg tggtctttgg aaacagaggt cgaaggaaag taaggagctg |
| 6781 | agagctcaca ttcataggtg ccgccagcct tcgtgcatct tcttgcatca tctctaagga |
| 6841 | gctcctctaa ttacaccatg cccgtcaccc catgagggat cagagaaggg atgagtcttc |
| 6901 | taaactctat attcgctgtg agtccaggtt gtaaggggga gcactgtgga tgcatcctat |
| 6961 | tgcactccag ctgatgacac caaagcttag gtgtttgctg aaagttcttg atgttgtgac |
| 7021 | ttaccacccc tgcctcacaa ctgcagacat aaggggacta tggattgctt agcaggaaag |
| 7081 | gcactggttc tcaagggcgg ctgcccttgg gaatcttctg gtcccaacca gaaagactgt |
| 7141 | ggcttgattt tctcaggtgc agcccagccg tagggccttt tcagagcacc ccctggttat |
| 7201 | tgcaacattc atcaaagttt ctagaacctc tggcctaaag gaagggcctg gtgggatcta |
| 7261 | cttggcactc gctggggggc caccccccag tgccactctc actaggcctc tgattgcact |
| 7321 | tgtgtaggat gaagctggtg ggtgatggga actcagcacc tcccctcagg cagaaaagaa |
| 7381 | tcatctgtgg agcttcaaaa gaaggggcct ggagtctctg cagaccaatt caacccaaat |
| 7441 | ctcgggggct ctttcatgat tctaatgggc aaccagggtt gaaaccctta tttctagggt |
| 7501 | cttcagttgt acaagactgt gggtctgtac cagagccccc gtcagagtag aataaaaggc |
| 7561 | tgggtagggt agagattccc atgtgcagtg gagagaacaa tctgcagtca ctgataagcc |
| 7621 | tgagacttgg ctcatttcaa aagcgttcaa ttcatcctca ccagcagttc agctggaaag |
| 7681 | gggcaaatac ccccacctga gctttgaaaa cgccctggga ccctctgcat tctctaagta |
| 7741 | agttatagaa accagtctct tccctccttt gtgagtgagc tgctattcca cgtaggcaac |
| 7801 | acctgttgaa attgccctca atgtctactc tgcatttctt tcttgtgata agcacacact |
| 7861 | tttattgcaa cataatgatc tgctcacatt tccttgcctg ggggctgtaa aaccttacag |
| 7921 | aacagaaatc cttgcctctt tcaccagcca cacctgccat accaggggta cagctttgta |
| 7981 | ctattgaaga cacagacagg atttttaaat gtaaatctat ttttgtaact ttgttgcggg |
| 8041 | atatagttct ctttatgtag cactgaactt tgtacaatat atttttagaa actcattttt |
| 8101 | ctactaaaac aaacacagtt tactttagag agactgcaat agaatcaaaa tttgaaactg |
| 8161 | aaatctttgt ttaaaagggt taagttgagg caagaggaaa gccctttctc tctcttataa |
| 8221 | aaaggcacaa cctcattggg gagctaagct aggtcattgt catggtgaag aagagaagca |
| 8281 | tcgtttttat atttaggaaa ttttaaaaga tgatggaaag cacatttagc ttggtctgag |
| 8341 | gcaggttctg ttggggcagt gttaatggaa agggctcact gttgttacta ctagaaaaat |
| 8401 | ccagttgcat gccatactct catcatctgc cagtgtaacc ctgtacatgt aagaaaagca |
| 8461 | ataacatagc actttgttgg tttatatata taatgtgact tcaatgcaaa ttttattttt |
| 8521 | atatttacaa ttgatatgca tttaccagta taaactagac atgtctggag agcctaataa |
| 8581 | tgttcagcac actttggtta gttcaccaac agtcttacca agcctgggcc cagccaccct |
| 8641 | agagaagtta ttcagccctg gctgcagtga catcacctga ggagctttta aaagcttgaa |
| 8701 | gcccagctac acctcagacc gattaaacgc aaatctctgg ggctgaaacc caagcattcg |
| 8761 | tagtttttaa agctcctgag gtcattccaa tgtgcggcca aagttgagaa ctactggcct |
| 8821 | agggattagc cacaaggaca tggacttgga ggcaaattct gcaggtgtat gtgattctca |
| 8881 | ggcctagaga gctaagacac aaagacctcc acatctgtcg ctgagagtca agaacctgaa |
| 8941 | cagagtttcc atgaaggttc tccaagcact agaagggaga gtgtctaaac aatggttgaa |
| 9001 | aagcaaagga aatataaaac agacacctct ttccatttcc taaggtttct ctctttatta |
| 9061 | agggtggact agtaataaaa tataatattc ttgctgctta tgcagctgac attgttgccc |
| 9121 | tccctaaagc aaccaagtag cctttatttc ccacagtgaa agaaaacgct ggcctatcag |
| 9181 | ttacattaca aaaggcagat ttcaagagga ttgagtaagt agttggatgg ctttcataaa |
| 9241 | aacaagaatt caagaagagg attcatgctt taagaaacat ttgttataca ttcctcacaa |
| 9301 | attatacctg ggataaaaac tatgtagcag gcagtgtgtt ttccttccat gtctctctgc |
| 9361 | actacctgca gtgtgtcctc tgaggctgca agtctgtcct atctgaattc ccagcagaag |
| 9421 | cactaagaag ctccacccta tcacctagca gataaaacta tggggaaaac ttaaatctgt |
| 9481 | gcatacattt ctggatgcat ttacttatct ttaaaaaaaa aggaatccta tgacctgatt |
| 9541 | tggccacaaa aataatcttg ctgtacaata caatctcttg gaaattaaga gatcctatgg |
| 9601 | atttgatgac tggtattaga ggtgacaatg taaccgatta acaacagaca gcaataactt |
| 9661 | cgttttagaa acattcaagc aatagcttta tagcttcaac atatggtacg ttttaacctt |
| 9721 | gaaagttttg caatgatgaa agcagtattt gtacaaatga aaagcagaat tctcttttat |
| 9781 | atggtttata ctgttgatca gaaatgttga ttgtgcattg agtattaaaa aattagatgt |
| 9841 | atattattca ttgttcttta ctcctgagta ccttataata ataataatgt attctttgtt |
| 9901 | aacaa |

| SEQ IN NO: 17 | |
|---|---|
| MRPSGTAGAA LLALLAALCP ASRALEEKKV CQGTSNKLTQ LGTFEDHFLS LQRMFNNCEV | |
| VLGNLEITYV QRNYDLSFLK TIQEVAGYVL IALNTVERIP LENLQIIRGN MYYENSYALA | |
| VLSNYDANKT GLKELPMRNL QEILHGAVRF SNNPALCNVE SIQWRDIVSS DFLSNMSMDF | |
| QNHLGSCQKC DPSCPNGSCW GAGEENCQKL TKIICAQQCS GRCRGKSPSD CCHNQCAAGC | |
| TGPRESDCLV CRKFRDEATC KDTCPPLMLY NPTTYQMDVN PEGKYSFGAT CVKKCPRNYV | |
| VTDHGSCVRA CGADSYEMEE DGVRKCKKCE GPCRKVCNGI GIGEFKDSLS INATNIKHFK | |
| NCTSISGDLH ILPVAFRGDS FTHTPPLDPQ ELDILKTVKE ITGFLLIQAW PENRTDLHAF | |
| ENLEIIRGRT KQHGQFSLAV VSLNITSLGL RSLKEISDGD VIISGNKNLC YANTINWKKL | |
| FGTSGQKTKI ISNRGENSCK ATGQVCHALC SPEGCWGPEP RDCVSCRNVS RGRECVDKCN | |
| LLEGEPREFV ENSECIQCHP ECLPQAMNIT CIGRGPDNCI QCAHYIDGPH CVKTCPAGVM | |
| GENNTLVWKY ADAGHVCHLC HPNCTYGCTG PGLEGCPTNG PKIPSIATGM VGALLLLLVV | |
| ALGIGLFMRR RHIVRKRTLR RLLQERELVE PLTPSGEAPN QALLRILKET EFKKIKVLGS | |
| GAFGTVYKGL WIPEGEKVKI PVAIKELREA TSPKANKEIL DEAYVMASVD NPHVCRLLGI | |
| CLTSTVQLIT QLMPFGCLLD YVREHKDNIG SQYLLNWCVQ IAKGMNYLED RRLVHRDLAA | |
| RNVLVKTPQH VKITDFGLAK LLGAEEKEYH AEGGKVPIKW MALESILHRI YTHQSDVWSY | |
| GVTVWELMTF GSKPYDGIPA SEISSILEKG ERLPQPPICT IDVYMIMVKC WMIDADSRPK | |
| FRELIIEFSK MARDPQRYLV IQGDERMHLP SPTDSNFYRA LMDEEDMDDV VDADEYLIPQ | |
| QGFFSSPSTS RTPLLSSLSA TSNNSTVACI DRNGLQSCPI KEDSFLQRYS SDPTGALTED | |
| SIDDTFLPVP EYINQSVPKR PAGSVQNPVY HNQPLNPAPS RDPHYQDPHS TAVGNPEYLN | |
| TVQPTCVNST FDSPAHWAQK GSHQISLDNP DYQQDFFPKE AKPNGIFKGS TAENAEYLRV | |
| APQSSEFIGA | |
| R34G NRF2 sgRNA (SEQ ID NO: 18): | |
| cuuacuccaa gaucuauauc gcaccgacuc ggugccacuu uuucaaguug auaacggacu | 60 |
| agccuuauuu uaacuugcua uuucuagcuc uaaaac | 96 |

In some embodiments, the cancer gene is epidermal growth factor receptor (EGFR). EGFR is a transmembrane glycoprotein that is a member of the protein kinase superfamily. This protein is a receptor for members of the epidermal growth factor family. EGFR is a cell surface protein that binds to epidermal growth factor, thus inducing receptor dimerization and tyrosine autophosphorylation leading to cell proliferation. Mutations in this gene are associated with lung cancer. EGFR is a component of the cytokine storm which contributes to a severe form of COVID-19 resulting from infection with severe acute respiratory syndrome coronavirus-2 (SARS-CoV-2). In some embodiments, a de novo PAM (CTG→CGG) is created in EGFR by a L858R mutation (highlighted in SEQ ID NOs: 16 and 17 below).

```
SEQ ID NO: 16
      1 agacgtccgg gcagcccccg gcgcagcgcg gccgcagcag cctccgcccc ccgcacggtg
    61 tgagcgcccg acgcggccga ggcggccgga gtcccgagct agccccggcg gccgccgccg
  121 cccagaccgg acgacaggcc acctcgtcgg cgtccgcccg agtccccgcc tcgccgccaa
  181 cgccacaacc accgcgcacg gccccctgac tccgtccagt attgatcggg agagccggag
  241 cgagctcttc ggggagcagc gatgcgaccc tccgggacgg ccggggcagc gctcctggcg
  301 ctgctggctg cgctctgccc ggcgagtcgg gctctggagg aaaagaaagt ttgccaaggc
  361 acgagtaaca agctcacgca gttgggcact tttgaagatc attttctcag cctccagagg
  421 atgttcaata actgtgaggt ggtccttggg aatttggaaa ttacctatgt gcagaggaat
  481 tatgatcttt ccttcttaaa gaccatccag gaggtggctg gttatgtcct cattgccctc
  541 aacacagtgg agcgaattcc tttggaaaac ctgcagatca tcagaggaaa tatgtactac
  601 gaaaattcct atgccttagc agtcttatct aactatgatg caaataaaac cggactgaag
  661 gagctgccca tgagaaattt acaggaaatc ctgcatggcg ccgtgcggtt cagcaacaac
  721 cctgccctgt gcaacgtgga gagcatccag tggcgggaca tagtcagcag tgactttctc
  781 agcaacatgt cgatggactt ccagaaccac ctgggcagct gccaaaagtg tgatccaagc
  841 tgtcccaatg ggagctgctg gggtgcagga gaggagaact gccagaaact gaccaaaatc
  901 atctgtgccc agcagtgctc cgggcgctgc cgtggcaagt cccccagtga ctgctgccac
  961 aaccagtgtg ctgcaggctg cacaggcccc cgggagagcg actgcctggt ctgccgcaaa
1021 ttccgagacg aagccacgtg caaggacacc tgccccccac tcatgctcta caaccccacc
1081 acgtaccaga tggatgtgaa ccccgagggc aaatacagct ttggtgccac ctgcgtgaag
1141 aagtgtcccc gtaattatgt ggtgacagat cacggctcgt gcgtccgagc ctgtggggcc
1201 gacagctatg agatggagga agacggcgtc cgcaagtgta agaagtgcga agggccttgc
1261 cgcaaagtgt gtaacggaat aggtattggt gaatttaaag actcactctc cataaatgct
1321 acgaatatta aacacttcaa aaactgcacc tccatcagtg gcgatctcca catcctgccg
1381 gtggcattta ggggtgactc cttcacacat actcctcctc tggatccaca ggaactggat
1441 attctgaaaa ccgtaaagga aatcacaggg tttttgctga ttcaggcttg gcctgaaaac
1501 aggacggacc tccatgcctt tgagaaccta gaaatcatac gcggcaggac caagcaacat
1561 ggtcagtttt ctcttgcagt cgtcagcctg aacataacat ccttgggatt acgctccctc
1621 aaggagataa gtgatggaga tgtgataatt tcaggaaaca aaaatttgtg ctatgcaaat
1681 acaataaact ggaaaaaact gtttgggacc tccggtcaga aaaccaaaat tataagcaac
1741 agaggtgaaa acagctgcaa ggccacaggc caggtctgcc atgccttgtg ctcccccgag
1801 ggctgctggg gcccggagcc cagggactgc gtctcttgcc ggaatgtcag ccgaggcagg
1861 gaatgcgtgg acaagtgcaa ccttctggag ggtgagccaa gggagtttgt ggagaactct
1921 gagtgcatac agtgccaccc agagtgcctg cctcaggcca tgaacatcac ctgcacagga
1981 cggggaccag acaactgtat ccagtgtgcc cactacattg acggccccca ctgcgtcaag
2041 acctgcccgg caggagtcat gggagaaaac aacaccctgg tctggaagta cgcagacgcc
2101 ggccatgtgt gccacctgtg ccatccaaac tgcacctacg gatgcactgg gccaggtctt
2161 gaaggctgtc caacgaatgg gcctaagatc ccgtccatcg ccactgggat ggtgggggcc
2221 ctcctcttgc tgctggtggt ggccctgggg atcggcctct tcatgcgaag gcgccacatc
2281 gttcggaagc gcacgctgcg gaggctgctg caggagaggg agcttgtgga gcctcttaca
2341 cccagtggag aagctcccaa ccaagctctc ttgaggatct tgaaggaaac tgaattcaaa
2401 aagatcaaag tgctgggctc cggtgcgttc ggcacggtgt ataagggact ctggatccca
2461 gaaggtgaga aagttaaaat tcccgtcgct atcaaggaat taagagaagc aacatctccg
2521 aaagccaaca aggaaatcct cgatgaagcc tacgtgatgg ccagcgtgga caacccccac
2581 gtgtgccgcc tgctgggcat ctgcctcacc tccaccgtgc agctcatcac gcagctcatg
2641 cccttcggct gcctcctgga ctatgtccgg gaacacaaag acaatattgg ctcccagtac
2701 ctgctcaact ggtgtgtgca gatcgcaaag ggcatgaact acttggagga ccgtcgcttg
2761 gtgcaccgcg acctggcagc caggaacgta ctggtgaaaa caccgcagca tgtcaagatc
2821 acagattttg ggctggccaa actgctgggt gcggaagaga aagaatacca tgcagaagga
2881 ggcaaagtgc ctatcaagtg gatggcattg gaatcaattt tacacagaat ctatacccac
2941 cagagtgatg tctggagcta cggggtgact gtttgggagt tgatgacctt tggatccaag
3001 ccatatgacg gaatccctgc cagcgagatc tcctccatcc tggagaaagg agaacgcctc
3061 cctcagccac ccatatgtac catcgatgtc tacatgatca tggtcaagtg ctggatgata
3121 gacgcagata gtcgcccaaa gttccgtgag ttgatcatcg aattctccaa aatggcccga
3181 gacccccagc gctaccttgt cattcagggg gatgaaagaa tgcatttgcc aagtcctaca
3241 gactccaact tctaccgtgc cctgatggat gaagaagaca tggacgacgt ggtggatgcc
3301 gacgagtacc tcatcccaca gcagggcttc ttcagcagcc cctccacgtc acggactccc
3361 ctcctgagct ctctgagtgc aaccagcaac aattccaccg tggcttgcat tgatagaaat
3421 gggctgcaaa gctgtcccat caaggaagac agcttcttgc agcgatacag ctcagacccc
3481 acaggcgcct tgactgagga cagcatagac gacaccttcc tcccagtgcc tgaatacata
3541 aaccagtccg ttcccaaaag gcccgctggc tctgtgcaga atcctgtcta tcacaatcag
3601 cctctgaacc ccgcgcccag cagagaccca cactaccagg acccccacag cactgcagtg
3661 ggcaaccccg agtatctcaa cactgtccag cccacctgtg tcaacagcac attcgacagc
3721 cctgcccact gggcccagaa aggcagccac caaattagcc tggacaaccc tgactaccag
3781 caggacttct ttcccaagga agccaagcca aatggcatct ttaagggctc cacagctgaa
3841 aatgcagaat acctaagggt cgcgccacaa agcagtgaat ttattggagc atgaccacgg
3901 aggatagtat gagccctaaa aatccagact ctttcgatac ccaggaccaa gccacagcag
3961 gtcctccatc ccaacagcca tgcccgcatt agctcttaga cccacagact ggttttgcaa
4021 cgtttacacc gactagccag gaagtacttc cacctcgggc acattttggg aagttgcatt
4081 cctttgtctt caaactgtga agcatttaca gaaacgcatc cagcaagaat attgtccctt
4141 tgagcagaaa tttatctttc aaagaggtat atttgaaaaa aaaaaaaagt atatgtgagg
4201 atttttattg attggggatc ttggagtttt tcattgtcgc tattgatttt tacttcaatg
4261 ggctcttcca acaaggaaga agcttgctgg tagcacttgc taccctgagt tcatccaggc
4321 ccaactgtga gcaaggagca caagccacaa gtcttccaga ggatgcttga ttccagtggt
4381 tctgcttcaa ggcttccact gcaaaacact aaagatccaa gaaggccttc atggccccag
4441 caggccggat cggtactgta tcaagtcatg gcaggtacag taggataagc cactctgtcc
4501 cttcctgggc aaagaagaaa cggaggggat ggaattcttc cttagactta cttttgtaaa
4561 aatgtcccca cggtacttac tccccactga tggaccagtg gtttccagtc atgagcgtta
4621 gactgacttg tttgtcttcc attccattgt tttgaaactc agtatgctgc ccctgtcttg
4681 ctgtcatgaa atcagcaaga gaggatgaca catcaaataa taactcggat tccagcccac
4741 attggattca tcagcatttg gaccaatagc ccacagctga gaatgtggaa tacctaagga
4801 tagcaccgct tttgttctcg caaaaacgta tctcctaatt tgaggctcag atgaaatgca
4861 tcaggtcctt tggggcatag atcagaagac tacaaaaatg aagctgctct gaaatctcct
4921 ttagccatca ccccaacccc ccaaaattag tttgtgttac ttatggaaga tagttttctc
4981 cttttacttc acttcaaaag ctttttactc aaagagtata tgttccctcc aggtcagctg
5041 cccccaaacc ccctccttac gctttgtcac acaaaaagtg tctctgcctt gagtcatcta
5101 ttcaagcact tacagctctg gccacaacag ggcattttac aggtgcgaat gacagtagca
5161 ttatgagtag tgtggaattc aggtagtaaa tatgaaacta gggtttgaaa ttgataatgc
5221 tttcacaaca tttgcagatg ttttagaagg aaaaaagttc cttcctaaaa taatttctct
5281 acaattggaa gattggaaga ttcagctagt taggagccca ccttttttcc taatctgtgt
5341 gtgccctgta acctgactgg ttaacagcag tcctttgtaa acagtgtttt aaactctcct
5401 agtcaatatc caccccatcc aatttatcaa ggaagaaatg gttcagaaaa tattttcagc
5461 ctacagttat gttcagtcac acacacatac aaaatgttcc ttttgctttt aaagtaattt
5521 ttgactccca gatcagtcag agcccctaca gcattgttaa gaaagtattt gatttttgtc
5581 tcaatgaaaa taaaactata ttcatttcca ctctattatg ctctcaaata cccctaagca
5641 tctatactag cctggtatgg gtatgaaaga tacaaagata aataaaacat agtccctgat
5701 tctaagaaat tcacaattta gcaaaggaaa tggactcata gatgctaacc ttaaaacaac
5761 gtgacaaatg ccagacagga cccatcagcc aggcactgtg agagcacaga gcagggaggt
5821 tgggtcctgc ctgaggagac ctggaaggga ggcctcacag gaggatgacc aggtctcagt
5881 cagcggggag gtggaaagtg caggtgcatc aggggcaccc tgaccgagga aacagctgcc
5941 agaggcctcc actgctaaag tccacataag gctgaggtca gtcaccctaa acaacctgct
6001 ccctctaagc caggggatga gcttggagca tcccacaagt tccctaaaag ttgcagcccc
6061 cagggggatt ttgagctatc atctctgcac atgcttagtg agaagactac acaacatttc
6121 taagaatctg agattttata ttgtcagtta accactttca ttattcattc acctcaggac
6181 atgcagaaat atttcagtca gaactgggaa acagaaggac ctacattctg ctgtcactta
6241 tgtgtcaaga agcagatgat cgatgaggca ggtcagttgt aagtgagtca cattgtagca
6301 ttaaattcta gtatttttgt agtttgaaac agtaacttaa taaaagagca aaagctattc
6361 tagctttctt cttcatattt taattttcca ccataaagtt tagttgctaa attctattaa
6421 ttttaagatt gtgcttccca aaatagttct cacttcatct gtccagggag gcacagttct
6481 gtctggtaga agccgcaaag cccttagcct cttcacggat ctggcgactg tgatgggcag
6541 gtcaggagag gagctgccca aagtcccatg attttcacct aacagccctg atcagtcagt
6601 actcaaagct tggactccat ccctgaaggt cttcctgatt gatagcctgg ccttaatacc
6661 ctacagaaag cctgtccatt ggctgtttct tcctcagtca gttcctggaa gaccttaccc
6721 catgacccca gcttcagatg tggtctttgg aaacagaggt cgaaggaaag taaggagctg
6781 agagctcaca ttcataggtg ccgccagcct tcgtgcatct tcttgcatca tctctaagga
6841 gctcctctaa ttacaccatg cccgtcaccc catgagggat cagagaaggg atgagtcttc
6901 taaactctat attcgctgtg agtccaggtt gtaaggggga gcactgtgga tgcatcctat
6961 tgcactccag ctgatgacac caaagcttag gtgtttgctg aaagttcttg atgttgtgac
7021 ttaccacccc tgcctcacaa ctgcagacat aaggggacta tggattgctt agcaggaaag
7081 gcactggttc tcaagggcgg ctgcccttgg gaatcttctg gtcccaacca gaaagactgt
7141 ggcttgattt tctcaggtgc agcccagccg tagggccttt tcagagcacc ccctggttat
7201 tgcaacattc atcaaagttt ctagaacctc tggcctaaag gaagggcctg gtgggatcta
7261 cttggcactc gctggggggc caccccccag tgccactctc actaggcctc tgattgcact
7321 tgtgtaggat gaagctggtg ggtgatggga actcagcacc tcccctcagg cagaaaagaa
7381 tcatctgtgg agcttcaaaa gaaggggcct ggagtctctg cagaccaatt caacccaaat
7441 ctcgggggct ctttcatgat tctaatgggc aaccagggtt gaaaccctta tttctagggt
7501 cttcagttgt acaagactgt gggtctgtac cagagccccc gtcagagtag aataaaaggc
7561 tgggtagggt agagattccc atgtgcagtg gagagaacaa tctgcagtca ctgataagcc
7621 tgagacttgg ctcatttcaa aagcgttcaa ttcatcctca ccagcagttc agctggaaag
7681 gggcaaatac ccccacctga gctttgaaaa cgccctggga ccctctgcat tctctaagta
7741 agttatagaa accagtctct tccctccttt gtgagtgagc tgctattcca cgtaggcaac
7801 acctgttgaa attgccctca atgtctactc tgcatttctt tcttgtgata agcacacact
7861 tttattgcaa cataatgatc tgctcacatt tccttgcctg ggggctgtaa aaccttacag
7921 aacagaaatc cttgcctctt tcaccagcca cacctgccat accaggggta cagctttgta
7981 ctattgaaga cacagacagg atttttaaat gtaaatctat ttttgtaact ttgttgcggg
8041 atatagttct ctttatgtag cactgaactt tgtacaatat atttttagaa actcattttt
8101 ctactaaaac aaacacagtt tactttagag agactgcaat agaatcaaaa tttgaaactg
8161 aaatctttgt ttaaaagggt taagttgagg caagaggaaa gccctttctc tctcttataa
8221 aaaggcacaa cctcattggg gagctaagct aggtcattgt catggtgaag aagagaagca
8281 tcgtttttat atttaggaaa ttttaaaaga tgatggaaag cacatttagc ttggtctgag
8341 gcaggttctg ttggggcagt gttaatggaa agggctcact gttgttacta ctagaaaaat
8401 ccagttgcat gccatactct catcatctgc cagtgtaacc ctgtacatgt aagaaaagca
8461 ataacatagc actttgttgg tttatatata taatgtgact tcaatgcaaa ttttattttt
8521 atatttacaa ttgatatgca tttaccagta taaactagac atgtctggag agcctaataa
8581 tgttcagcac actttggtta gttcaccaac agtcttacca agcctgggcc cagccaccct
8641 agagaagtta ttcagccctg gctgcagtga catcacctga ggagctttta aaagcttgaa
8701 gcccagctac acctcagacc gattaaacgc aaatctctgg ggctgaaacc caagcattcg
8761 tagtttttaa agctcctgag gtcattccaa tgtgcggcca aagttgagaa ctactggcct
8821 agggattagc cacaaggaca tggacttgga ggcaaattct gcaggtgtat gtgattctca
8881 ggcctagaga gctaagacac aaagacctcc acatctgtcg ctgagagtca agaacctgaa
8941 cagagtttcc atgaaggttc tccaagcact agaagggaga gtgtctaaac aatggttgaa
9001 aagcaaagga aatataaaac agacacctct ttccatttcc taaggtttct ctctttatta
9061 agggtggact agtaataaaa tataatattc ttgctgctta tgcagctgac attgttgccc
9121 tccctaaagc aaccaagtag cctttatttc ccacagtgaa agaaaacgct ggcctatcag
9181 ttacattaca aaaggcagat ttcaagagga ttgagtaagt agttggatgg ctttcataaa
9241 aacaagaatt caagaagagg attcatgctt taagaaacat ttgttataca ttcctcacaa
9301 attatacctg ggataaaaac tatgtagcag gcagtgtgtt ttccttccat gtctctctgc
9361 actacctgca gtgtgtcctc tgaggctgca agtctgtcct atctgaattc ccagcagaag
9421 cactaagaag ctccacccta tcacctagca gataaaacta tggggaaaac ttaaatctgt
9481 gcatacattt ctggatgcat ttacttatct ttaaaaaaaa aggaatccta tgacctgatt
9541 tggccacaaa aataatcttg ctgtacaata caatctcttg gaaattaaga gatcctatgg
9601 atttgatgac tggtattaga ggtgacaatg taaccgatta acaacagaca gcaataactt
9661 cgttttagaa acattcaagc aatagcttta tagcttcaac atatggtacg ttttaacctt
9721 gaaagttttg caatgatgaa agcagtattt gtacaaatga aaagcagaat tctcttttat
9781 atggtttata ctgttgatca gaaatgttga ttgtgcattg agtattaaaa aattagatgt
9841 atattattca ttgttcttta ctcctgagta ccttataata ataataatgt attctttgtt
9901 aacaa
SEQ ID NO: 17
MRPSGTAGAA LLALLAALCP ASRALEEKKV CQGTSNKLTQ LGTFEDHFLS LQRMFNNCEV
VLGNLEITYV QRNYDLSFLK TIQEVAGYVL IALNTVERIP LENLQIIRGN MYYENSYALA
VLSNYDANKT GLKELPMRNL QEILHGAVRF SNNPALCNVE SIQWRDIVSS DFLSNMSMDF
QNHLGSCQKC DPSCPNGSCW GAGEENCQKL TKIICAQQCS GRCRGKSPSD CCHNQCAAGC
TGPRESDCLV CRKFRDEATC KDTCPPLMLY NPTTYQMDVN PEGKYSFGAT CVKKCPRNYV
VTDHGSCVRA CGADSYEMEE DGVRKCKKCE GPCRKVCNGI GIGEFKDSLS INATNIKHFK
NCTSISGDLH ILPVAFRGDS FTHTPPLDPQ ELDILKTVKE ITGFLLIQAW PENRTDLHAF
ENLEIIRGRT KQHGQFSLAV VSLNITSLGL RSLKEISDGD VIISGNKNLC YANTINWKKL
FGTSGQKTKI ISNRGENSCK ATGQVCHALC SPEGCWGPEP RDCVSCRNVS RGRECVDKCN
LLEGEPREFV ENSECIQCHP ECLPQAMNIT CTGRGPDNCI QCAHYIDGPH CVKTCPAGVM
GENNTLVWKY ADAGHVCHLC HPNCTYGCTG PGLEGCPTNG PKIPSIATGM VGALLLLLVV
ALGIGLFMRR RHIVRKRTLR RLLQERELVE PLTPSGEAPN QALLRILKET EFKKIKVLGS
GAFGTVYKGL WIPEGEKVKI PVAIKELREA TSPKANKEIL DEAYVMASVD NPHVCRLLGI
CLISTVQLIT QLMPFGCLLD YVREHKDNIG SQYLLNWCVQ IAKGMNYLED RRLVHRDLAA
RNVLVKTPQH VKITDFGLAK LLGAEEKEYH AEGGKVPIKW MALESILHRI YTHQSDVWSY
GVTVWELMTF GSKPYDGIPA SEISSILEKG ERLPQPPICT IDVYMIMVKC WMIDADSRPK
FRELIIEFSK MARDPQRYLV IQGDERMHLP SPTDSNFYRA LMDEEDMDDV VDADEYLIPQ
QGFFSSPSTS RTPLLSSLSA TSNNSTVACI DRNGLQSCPI KEDSFLQRYS SDPTGALTED
SIDDTFLPVP EYINQSVPKR PAGSVQNPVY HNQPLNPAPS RDPHYQDPHS TAVGNPEYLN
TVQPTCVNST FDSPAHWAQK GSHQISLDNP DYQQDFFPKE AKPNGIFKGS TAENAEYLRV
APQSSEFIGA
R34G NRF2 sgRNA (SEQ ID NO: 18):
cuuacuccaa gaucuauauc gcaccgacuc ggugccacuu uuucaaguug auaacggacu 60
agccuuauuu uaacuugcua uuucuagcuc uaaaac 96
```

### EXAMPLES

The present disclosure is further defined in the following Examples. It should be understood that these Examples, while indicating exemplary embodiments of the present disclosure, are given by way of illustration only.

### Example 1

Biodistribution of Adenovirus after Subcutaneous or Intratumoral Injection in a Human Xenograft Mouse Model of H1703 Non-Small Cell Lung Cancer (NSCLC)

Biodistribution of adenovirus after intratumoral injection is determined in mice implanted with wildtype H1703 squamous non-small cell lung carcinoma cells. The H1703 cells are implanted into the mice either subcutaneously, or orthoto pically (i.e., in lung tissue), as described below.

### Subcutaneous Injection of H1703 Cells

Subcutaneous injections of H1703 cells are performed on 5 female athymic nude mice and 5 female NCG mice at age 6 weeks. This SubC injection implants tumor cells into the left flank of the fatty part of the belly. These cells are injected to grow into solid mass tumors in the murine model. The H1703 human tumor-derived cell line is injected at a concentration of 4×10⁶ in a total volume of 200 μL of sterile PBS. This injection is performed while restraining the mouse using the researcher's non-dominant hand. After injection, the mouse is returned to its home cage with ad libitum access to food and water as usual. As the cells proliferate, the tumors are measured using calipers at 3 day intervals until tumor volume has reached at least 1.5 cm in any dimension. If tumors grow beyond 1.5 cm in any dimension, the mouse is humanely euthanized using CO₂ inhalation. The purpose of this experiment is to determine suitability of tumor growth between each strain of mouse (athymic nude or NCG). Athymic nude or NCG mice are selected for the rest of the experiments depending on the success of tumor growth for each strain.

Mouse body weights are collected every 3 days to monitor animal health. If the mouse has more than 20% body weight loss at any time during this or the following experiments, the mouse is euthanized appropriately and according to the guidelines of this protocol.

TABLE 2
Animals for evaluation by subcutaneous injection

| | | treatment |
|---|---|---|
| | saline | with H1703 |
| Group | control | cancer cells |
| Athymic Nude | 5 | 5 |
| NCG | 5 | 5 |
| total | | 20 |
| requested | | |

### Orthotopic Injection of H1703 Cells

H1703 cells are administered orthotopically to 5 female mice of either of the previously determined strains at age 6 weeks using a surgery to access the lung on the dorsal side. The H1703 tumor-derived cells are administered at a concentration of 1×10⁶ in a total volume of 40 μL of sterile PBS. This will be performed under isoflurane anesthesia. Animal weights are collected the day of surgery and every 3 days thereafter. After injection, the mouse is returned to its home cage with ad libitum access to food and water as usual. The tumors are measured using either IVIS bioluminescence or MRI imaging at 3 day intervals until tumor volume has reached more than 1.5 cm in any dimension. If tumors grow beyond 1.5 cm in any dimension, the mouse is humanely euthanized using CO₂ inhalation. The purpose of this experiment is to determine suitability of tumor growth in an orthotopic model using either one of the optimized strains of mouse established in the previous experiments (athymic nude or NCG).

TABLE 3
Animals requested for orthotopic injection

| | Mock | Surgical Injection |
|---|---|---|
| | Surgery | with H1703 |
| Group | Control | cancer cells |
| NCG or Athymic | 5 | 5 |
| Nude | | |
| Total Requested | | 10 |

The tumors are grown to a size of approximately 100 mm³ before injection of adenovirus. Adenovirus engineered to express luciferase under transcriptional control of a CMV promoter are administered by single injection into 4 different quadrants of the tumor. The treatment groups are described in the table below.

TABLE 4
Treatment groups

| Tumor | Virus | Total | Mouse |
|---|---|---|---|
| Type | (PFU) | volume | number |
| H1703 WT | 1 × 10⁸ | 80 μL | 20 |
| | 1 × 10⁹ | 80 μL | 20 |
| | PBS | 80 μL | 16 |

Mice are live imaged (ventrally and laterally) for luciferase activity with a IVIS luminescence camera. Five minutes before imaging, 100 ul of sodium luciferin (1.58 mg/ml; 10 μg/g body weight) is injected intraperitoneally. In vivo imaging for luciferase expression using IVIS is performed 1d, 2d, 3d, 6d, 10d, 15d, and 18d post viral injection and body weights are recorded.

Cohorts of 5 mice for Ad-CMV-Luc (or 4 mice for PBS) are euthanized, and tumor and peripheral tissues (lung, liver, blood, spleen, thymus, pancreas, stomach, intestine, kidneys, heart, ovaries, brain) are collected at 1d, 3d, 6d and 18d post viral injection timepoints. Tumor cell identity is sequence verified.

In vivo Bioluminescence Imaging (IVIS) is calculated as photon flux or RLU (In) for each viral PFU tested.

For viral genome quantification, DNA from tumor and peripheral tissues is isolated using the Dneasy blood and tissue kit (Qiagen 69504). Quantification of viral genome in tissue and tumor specimens is performed by real-time PCR using Adeno-X qPCR Titration kit (Cat #632252).

Luciferase transgene expression in tissues is determined by lysing cells from tumor and peripheral tissues using Promega lysis buffer. Luciferase gene expression is measured using the Bright-Glo Luciferase Assay system (Promega, E2620).

Liver cytotoxicity is determined by measuring serum ALT (alanine aminotransferase) and AST (aspartate aminotransferase) using ELISA to account for virus-induce damage to hepatocytes.

### Example 2

### Dosing and In Vivo Efficacy for Treatment of Subcutaneous or Lung Tumors

The treatment window is based on the tumor growth curve established for subcutaneous injection in Example 1 above. It is estimated to be between 4-6 weeks after the SubC tumor cell injection. As performed in Example 2, an identical subcutaneous injection will deliver the Adenovirus at 2×10⁸ MOI in 100 μL or AAV at 1×10¹¹ MOI in 100 μL directly into the tumor mass once the size has reached 80-100 mm³. If this dose is not sufficient to illicit changes in chemotherapy response, doses are adjusted. For orthotopic injection, the therapeutic window is determined based on the tumor growth curve established from previous experiments. Using a second minor surgery, either adenovirus or AAV is injected directly into the lung tumor. Groups of animals to be used for each type of injection are shown in Table 5 below.

TABLE 5
Animals to be used for dosing

| Dose | saline | AAV | Adenovirus |
|---|---|---|---|
| low | — | 5 | 5 |
| med | 5 | 5 | 5 |
| high | — | 5 | 5 |
| Total requested | | | 35 |

The effectiveness of the CRISPR-directed intervention is also tested in combination with chemotherapy treatment. Cisplatin is delivered via intravenous tail vein injection every 3 days for 10 days at a dose of 3 mg/kg for 4 total cycles. Tumor size is evaluated using calipers. Various formulations of targeted gene-editing tools and additional dose ranges are tested to optimize treatment efficacy.

### Example 3

Evaluation of Adenovirus Serotype 5 Vectors Encoding One or Three sgRNAs Targeting R34G NRF2 in Lung Cancer Cells, and Comparison of a Single Adenovirus 5 Vector Encoding Cas9 and R34G NRF2 sgRNAs Versus a Dual Vector System Containing Separate Adenoviral Vectors Encoding Cas9 or Three R34G NRF2 sgRNAs in Lung Cancer Cells

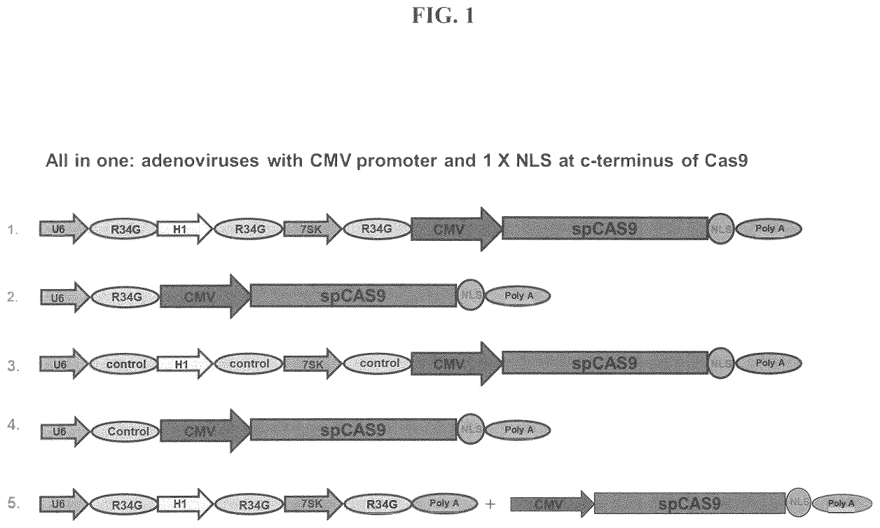

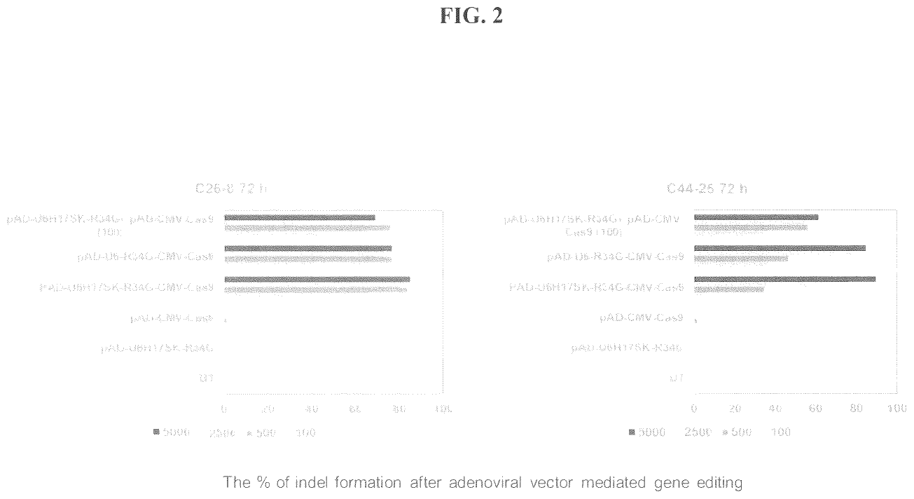

A schematic of the adenovirus serotype 5 vectors evaluated in this study is provided in FIG. 1. Treatment group 1 was a single vector encoding one copy of an R34G NRF2 sgRNA (SEQ ID NO:18) driven by the U6 promoter, and treatment group 2 was a single vector encoding three copies of the R34G NRF2 sgRNA driven by the U6, H1 or 7SK promoter. Both of these vectors also encoded wildytpe Streptococcus pyogenes Cas9 (spCas9) driven by the CMV promoter, with one copy of an NLS fused within the C-terminus of spCas9. The spCas9 cassette was obtained from SignaGen Laboratories. Treatment groups 3 and 4 were essentially the same as 1 and 2, except that the 20 base pairs encoding the DNA binding domain of the R34G NRF2 gRNA was replaced with a scrambled DNA sequence as a negative control. Treatment group 5 was a dual vector system with one vector encoding 3 copies of the R34G NRF2 gRNAs, but lacking a spCas9 cassette; and the other vector containing an spCas9 cassette comprising a CMV promoter, but lacking a gRNA cassette.

FIG. 2 shows the % of indels after lung cancer cells infected with different adenoviral vectors at different MOI PFU/ml, e.g., MOI at 100, 500, 2500, and 5000. 72 h after infection, gDNA were collected and subjected to Sanger sequencing. The % of indels was analyzed using DECODR. In FIG. 43, the % of indels was presented with lung cancer C26-8 cells on the left, and lung cancer C44-25 cells on the right. Both of these squamous non-small cell lung carcinoma cell lines were derived from the parental H1703 cell line by engineering them to contain a homozygous R34G mutation in the NRF2 gene. From the bottom to the top of each figure, the first group is labeled with UT, indicating the cells were not treated (negative control). For the 2nd group from the bottom, the cells were transduced with pAD-U6H17SK-R34G only (negative control), so, no indel formation was observed. For the 3rd group labeled as pAD-CMV-Cas9, only Cas9 was introduced (negative control), so there was no indel formation, or at background levels. The 4th group from the bottom was pAD-U6H17SK-R34G-CMV-Cas9 (three copies of the sgRNA, plus Cas9), in which we observed increased levels of indel formation relative to the negative controls. The 5th group from the bottom was pAD-U6-R34G-CMV-Cas9 (one copy of the sgRNA, plus Cas 9) infected cells. We observed similar levels of indel formation for this 5th group containing one copy of the sgRNA as the 4th group containing three copies of the sgRNA. The cells in the 6th group were infected with dual vectors, where one vector provided 3 copies of the R34G NRF2 sgRNA, while the spCas9 was introduced by a separate pAD-CMV-Cas9 vector. This 6th group resulted in similar levels of indel formation as the 4th and 5th groups.

In summary, vectors with one copy or three copies of R34G gRNAs showed similar gene editing efficacy in the two lung cancer cell lines transduced. Furthermore, the all in one vector performed similarly as the dual vector or better in terms of gene editing.

### Example 4

### Comparison of Transduction Efficacy in Lung Cancer Cells of AAV6 and Adenovirus

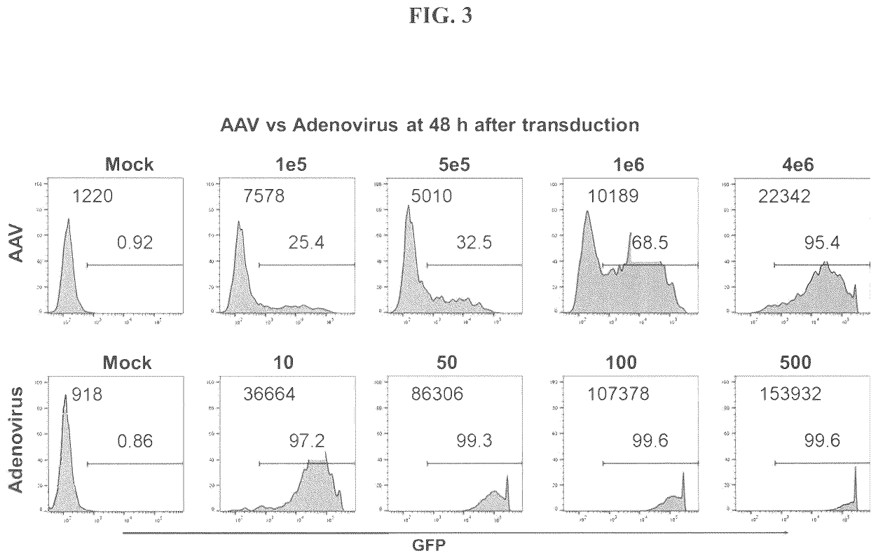

To compare the transduction efficacy in lung cancer cells between AAV6 and adenoviruses, we transduced lung cancer cells with both vectors respectively expressing GFP and evaluated the extent of GFP expression via flow cytometry at 48 h post-transduction. As shown in FIG. 3, as low as 10 multiplicity of infection (MOI) of adenovirus efficiently transduced the cells (lower panel) which outperformed the highest MOI of 4 million in AAV6 tested (upper panel). The numbers right above the scale bars represented the percentage of GFP positive cells, and the numbers at the left corners of each image indicated the mean fluorescent intensity (MFI) of GFP. The data demonstrate that the adenoviral vector has greater transduction efficiency for lung cancer cells relative to the AAV6 vector.

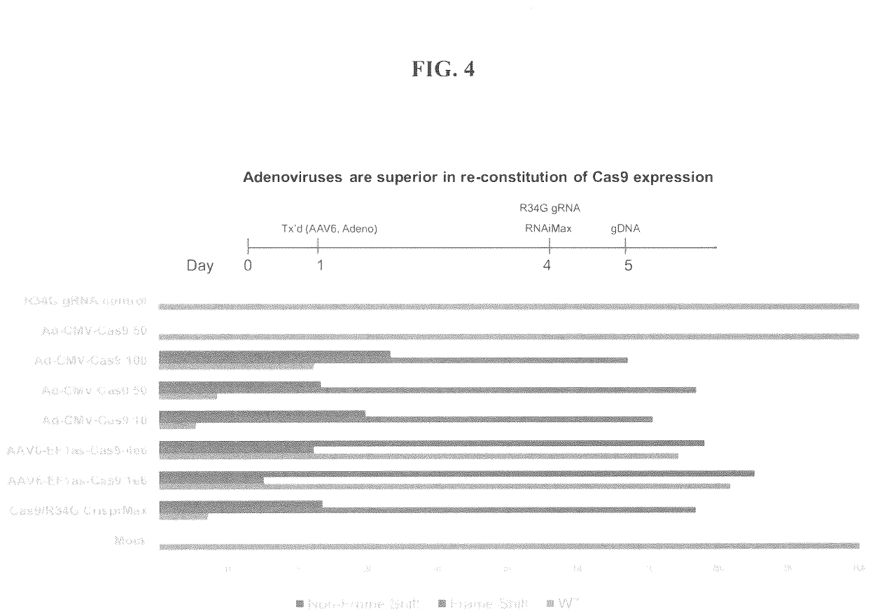

Since the size of GFP is only a fraction of the size of the Cas9 protein, we also compared the potential to use AAV6 and adenovirus to express Cas9 directly in lung cancer cells. The experiment was split into steps. In the first step, the cells were treated with nucleic acids encoding Cas9 for four days. The nucleic acids endocing Cas9 were delivered by AAV6 or adenoviral vector. In the second step, sufficient LNPs containing R34G sgRNA was delivered 4 days after infection with the AAV6 or adenovirus. The cells were transfected with R34G gRNA with the reagent RNAiMax which was purchased from Thermo Fisher Scientific. At Day 5 the gDNA from these cells was collected, and the PCR amplicons flanking the R34G mutation were Sanger sequenced. The treatment groups in the order shown in FIG. 4 (top to bottom) are described below.

R34G gRNA control: Cells treated with R34G gRNA, but not with a Cas9 construct (negative control).

Ad-CMV-Cas9 50: Cells treated with adenovirus encoding Cas9 under transcription control of the CMV promoter at an MOI of 50, but not treated with a gRNA construct (negative control).

Ad-CMV-Cas9 100: Cells treated with 1) adenovirus encoding Cas9 under transcription control of the CMV promoter at an MOI of 100, and 2) LNP mediated R34G sgRNA at day 4.

Ad-CMV-Cas9 50: Cells treated with 1) adenovirus encoding Cas9 under transcription control of the CMV promoter at an MOI of 50, and 2) LNP mediated R34G sgRNA at day 4.

Ad-CMV-Cas9 10: Cells treated with 1) adenovirus encoding Cas9 under transcription control of the CMV promoter at an MOI of 10, and 2) LNP mediated R34G sgRNA at day 4.

AAV6-EF1as-Cas9-4e6: Cells treated with 1) AAV6 encoding Cas9 under transcription control of the core EF1alpha short (as) promoter at an MOI of 4×10⁶, and 2) LNP mediated R34G sgRNA at day 4.

AAV6-EF1as-Cas9-1e6: Cells treated with 1) AAV6 encoding Cas9 under transcription control of the core EF1alpha short (as) promoter at an MOI of 1×10⁶, and 2) LNP mediated R34G sgRNA at day 4.

Cas9/R34G CrisprMax™: Cells treated with 1) LNPs containing Cas9 protein (CrisprMax™), and 2) LNP mediated R34G sgRNA at day 4 (positive control).

Mock: Cells not treated with a Cas9 construct or an sgRNA construct (negative control).

As shown in FIG. 4, adenoviral vector was superior in the re-constitution of Cas9 expression relative to the AAV6 vector where Cas9 expression was driven by the core EF1alpha short promoter, and further evidenced by the overall gene editing efficacy. The reason the EF1apha short promoter was chosen for the AAV vector to express Cas9 is that the AAV vector does not have the size capacity to contain the full CMV promoter. The data indicated that with the use of the AAV6 with multiplicity of infection (MOI) as high as 4 million, 70-80% of cells remained un-edited, while with the adenoviral vector with MOI as low as 10, only 10% of cells remained un-edited, i.e., more than 90% of the cells were gene edited with the adenoviral vector. In this experiment, the R34G sgRNA was designed based upon the R34G mutation in the human NEF2L2 gene, which encodes the NRF2 protein. These results demonstrate that adenoviral vectors have much greater efficacy than AAV vectors for expressing Cas9 in lung cancer cells.

### Example 5

### Athymic Nude Mice Background:

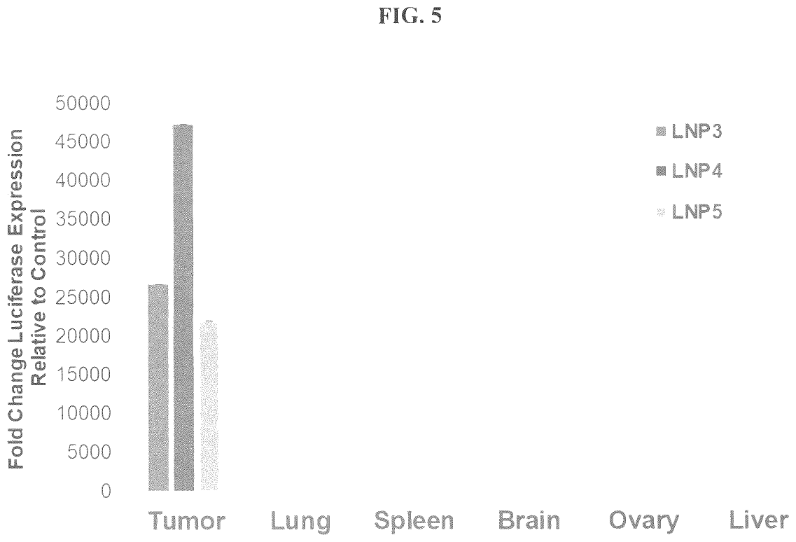

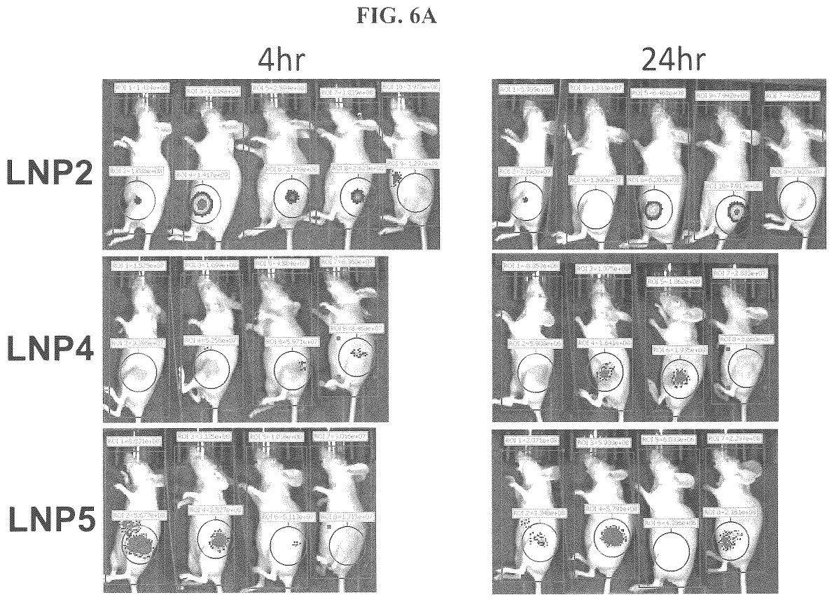

Athymic nude mice are commonly used for cancer research because of their immunodeficient background. These mice have homozygous mutations in the Foxn1 gene that causes a deteriorated or absent thymus. T-lymphocytes, mature in the thymus and are responsible for host graft reaction. Therefore, the absence of a thymus in this mouse model allows for tumor growth and development when human cancer cells are injected subcutaneously. Another advantage of this model is that the tumor can be easily observed since the mice do not develop hair. Relative luciferase expression is graphed as fold change relative to control/un-injected mice (FIG. 5). Data show no biodistribution to the lungs, spleen, brain, ovaries, or liver but 25,000 to 45,000 fold expression in the injected tumor samples for LNP3, 4 and 5.

Biodistribution of LNP/fLuc Intratumoral Delivery (qRTPCR) to H1703 (44-25) SubQ Developed in NCG Mice Background

### Materials and Methods

In order to analyze the biodistribution of the luciferase expressing LNPs, quantitative PCR was done. Briefly, RNA was extracted from tissue samples using the TRIzol RNA extraction protocol as described by the manufacturer (Invitrogen). Complementary DNA (cDNA) was then synthesized using Applied Biosystems High-Capacity RNA-to-cDNA kit at 250 ng per reaction and qPCR was performed in triplicate with Fast SYBR Green Master Mix was used. Table 6 shows the primer sequences that were used. Samples were run on a Bio-Rad CFX384 real time PCR detection system according to the manufactures conditions: 95° for 20 sec followed by 40 cycles of 95° for 3° seconds and 60° for 30 seconds. Relative quantification for each transcript was obtained by normalizing against Gapdh transcript abundance according to the formula 2^{(-Ct)}/2^{(CtGapdh)}.

TABLE 6
Primer sequences for qPCR analysis. Gapdh-mouse primers used
for lung, ovary, liver, spleen and brain tissues. Gapdh-human
primers used for tumor tissues.

| Primer name | Sequence 5′-3′ | SEQ ID NO |
|---|---|---|
| Gapdh-mouse F | CAT GGC CTT CCG TGT TCC TA | 19 |
| Gapdh-mouse R | ACT TGG CAG GTT TCT CCA GG | 20 |
| Gapdh-human F | TCTCCTCTGACTTCAACAGCGAC | 21 |
| Gapdh-human R | CCCTGTTGCTGTAGCCAAATTC | 22 |
| Luciferase LNP-F | TGATGTACCGGTTCGAGGAG | 23 |
| Luciferase LNP-R | TTGTCGATCAGGGTGCTCTT | 24 |

### Example 6

Intratumoral Delivery of LNP/fLuc (4h, 24h IVIS Data) to H1703 (44-25) subQ Developed in the Nude Mice

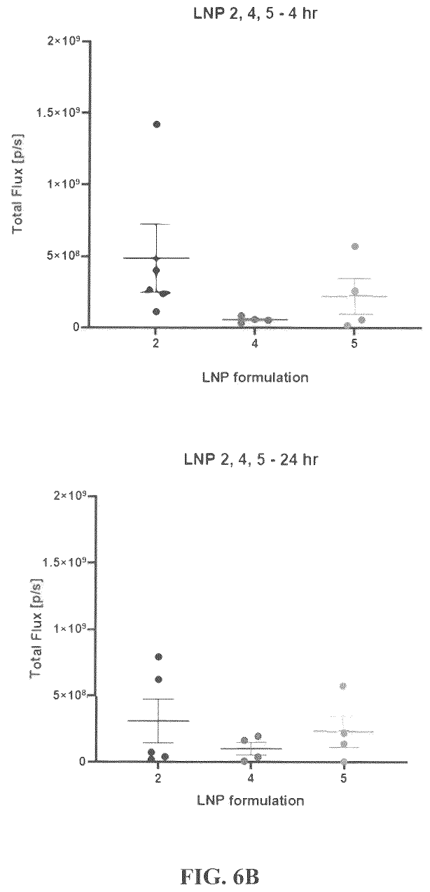

The goal of this experiment is to visualize the expression of luciferase mRNA after intratumoral injection. Lipid nanoparticles containing luciferase mRNA were injected into the subcutaneous tumor of athymic nude mice. Images were taken 4 and 24 hours after injection. FIG. 6 shows all of LNP/fLuc delivered directly to the tumor led to the expression of the fLuc gene in the tumor region.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old athymic nude female mice (Charles River) by subcutaneous implantation of 5×10⁶ cells (human lung squamous cell-derived H1703 clone 44-25) in BD matrigel (volume ratio 1:1). Tumor growth was measured and estimated using a caliper and calculated as volume (mm³)=(length [mm]×(width [mm])²×0.5. When tumors reached ˜60-150 mm³, mice were separated to experimental groups. Intratumoral injections consisted of 2 μg of LNP in a total volume of 25 μL purchased from Precision Nanosystems.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 7

Delivery of Adenoviral Vector (fLuc) in H1703 (44-25) Subcutaneous Xenograft Model

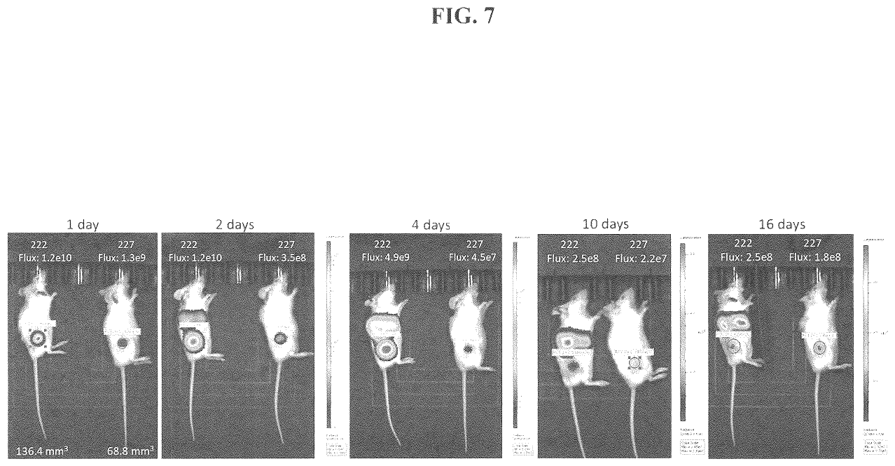

Adenovirus containing the firefly luciferase transgene was used to assess intratumoral delivery and expression within a xenograft mouse model. CRISPR-engineered human lung squamous cell carcinoma cell line, H1703 44-25, was implanted subcutaneously. Once tumors reached 60-150 mm³ in size, mice were injected intratumorally with 1.5e⁹ pfu of Ad5-CMV-fLuc (SignaGen Laboratories) to 3 sites within the tumor. Bioluminescence imaging was conducted on mice 1-, 2-, 4-, 10- and 16-days post injection. FIG. 7 displays time course of the bioluminescent signal post intratumoral injection. Signal from each tumor is quantified by establishing a region of interest (ROI) of the tumor. The ROI value listed as total flux photons of each tumor. One day post intratumoral injection in both mice with different sized tumors created a strong signal with comparable ROIs. The signal increased and plateaued over the course of 16 days.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of 5×10⁶ cells (human lung squamous cell-derived H1703 clone 44-25) in BD matirgel (volume ratio 1:1). Tumor growth was measured and estimated using a caliper and calculated as volume (mm³)=(length [mm]×(width [mm])²×0.5. When tumors reached ˜60-150 mm³, mice were separated to experimental groups. Adenoviral injections consisted of 1.5e⁹ pfu in a total volume of 30 μL of Ad5-CMV-fLuc purchased from SignaGen Laboratories.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% Isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 8

### Delivery of Adenoviral Vector Targeting Nrf2 in H1703 (44-25) Subcutaneous Xenograft Model

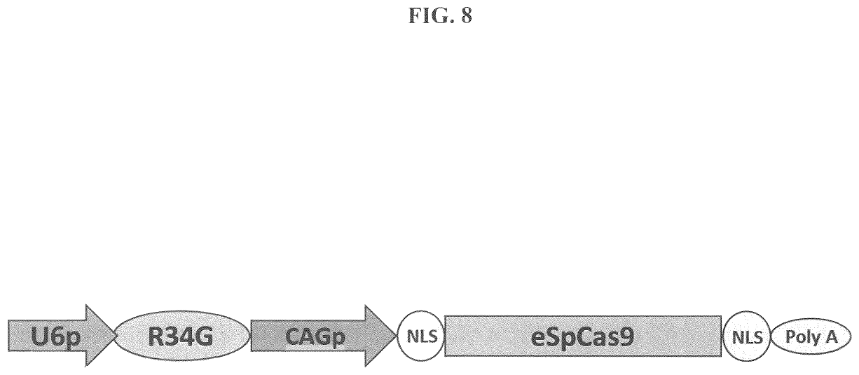

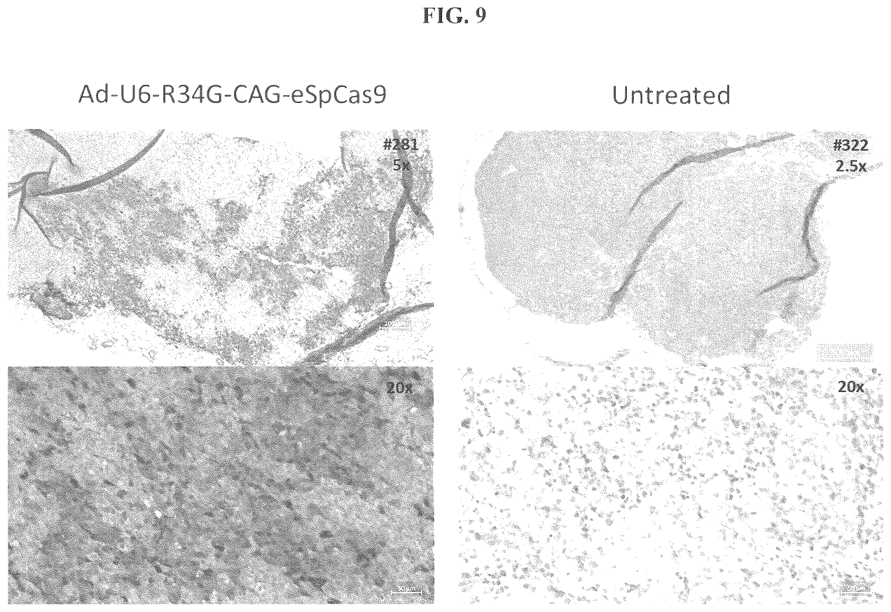

Adenovirus containing CRISPR/Cas9 targeting NRF2 was used to assess intratumoral delivery and expression within a xenograft mouse model. CRISPR-engineered human lung squamous cell carcinoma cell line, H1703 44-25, was implanted subcutaneously. Once tumors reached 60-150 mm³ in size, mice were injected intratumorally with 3.6e⁹ pfu of Ad-U6-R34G-CAG-eSpCas9 or without any treatment. FIG. 8 depicts the schematic diagram of Ad-U6-R34G-CAG-eSpCas9 used with U6 promoter driving R34G-targeting sgRNA (SEQ ID NO:25) expression. Forty-eight hours post intratumoral injection, tumors were collected for immunohistochemistry staining and analysis. Immunostained tumor tissues were imaged and analyzed for localization and expression of Cas9. As shown in FIG. 9, the top panel of images displays 5× and 2.5× magnification of treated and untreated tumors followed by 20× magnification in the bottom panel of images. DAB staining was utilized to visualize localization of the signal within the tumor section. As seen in Ad-U6-R34G-CAG-eSpCas9 treated tumor tissues, there is signal (dark brown reaction) throughout the tumor section as opposed to the clear, even image obtained from untreated tumor sections.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of 5×10⁶ cells (human lung squamous cell-derived H1703 clone 44-25) in BD matirgel (volume ratio 1:1). Tumor growth was measured and estimated using a caliper and calculated as volume (mm³)=(length [mm]×(width [mm])²×0.5. When tumors reached ˜60-150 mm³, mice were separated to experimental groups. Adenoviral injections consisted of 3.6e⁹ pfu in a total volume of 30 μL of Ad-U6-R34G-CAG-eSpCas9 purchased from Vector Biolabs.

### Immunohistochemistry

Tumor tissues were fixed with 4% paraformaldehyde in 1×PBS overnight at 4° C. Cas9 labelling was performed using immunohistochemistry on fixed tumor tissues. Tumors were frozen and sliced at 10 μm and mounted directly to slides. After blocking with 5% Normal Goat Serum (Vector), rabbit anti-Cas9 primary antibody (Abcam) was applied directly to each section in incubation buffer (1% BSA in PBS) at a 1:100 dilution ratio. The primary antibody was incubated overnight at 4° C. in humidity chambers to prevent drying. Goat anti-rabbit biotinylated secondary (Vector) was applied directly to each section in incubation buffer at a 1:200 dilution ratio. The secondary antibody incubated at room temperature for 1 hour. ABC Elite HRP kit (Vector) was used for detection with methods following the kit's protocol. DAB was used to develop the stain and slides were counterstained with Hemotoxylin. Slides were then dehydrated in ethanol/xylene and permanently mounted with Acrytol. Slides were imaged on LEICA DMIL LED microscope.

### Example 9

### Delivery of Adenoviral Vector Targeting Nrf2 in NCI PDMR PDX

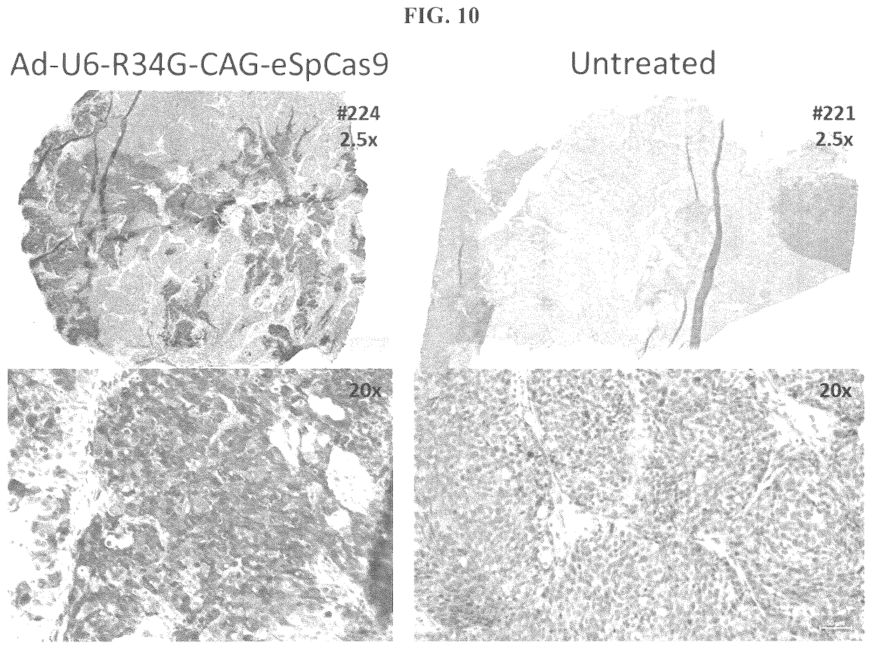

Adenovirus containing CRISPR/Cas9 targeting NRF2 was used to assess intratumoral delivery and expression within a patient-derived xenograft mouse model. Human lung squamous cell carcinoma tumor fragments (NCI PMDR) were implanted subcutaneously. Once tumors reached 60-150 mm³ in size, mice were injected intratumorally with 3.6e⁹ pfu of Ad-U6-R34G-CAG-eSpCas9 (schematic diagram shown in FIG. 8) or without any treatment. Forty-eight hours post intratumoral injection, tumors were collected for immunohistochemistry staining and analysis. Immunostained tumor tissues were imaged and analyzed for localization and expression of Cas9. As shown in FIG. 10, the top panel of images displays 2.5× magnification of treated and untreated tumors followed by 20× magnification in the bottom panel of images. DAB staining was utilized to visualize localization of the signal within the tumor section. As seen in Ad-U6-R34G-CAG-eSpCas9 treated tumor tissues, there is signal (dark brown reaction) throughout the tumor section as opposed to the clear, even image obtained from untreated tumor sections.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of PDX fragments derived from human lung squamous cell carcinoma tumor (obtained from NCI PDMR, specimen ID 073-R). Tumor growth was measured and estimated using a caliper and calculated as volume (mm³)=(length [mm]×(width [mm])²×0.5. When tumors reached ˜60-150 mm³, mice were separated to experimental groups. Adenoviral injections consisted of 3.6e⁹ pfu in a total volume of 30 μL of Ad-U6-R34G-CAG-eSpCas9 purchased from Vector Biolabs.

### Immunohistochemistry

Tumor tissues were fixed with 4% paraformaldehyde in 1×PBS overnight at 4° C. Cas9 labelling was performed using immunohistochemistry on fixed tumor tissues. Tumors were frozen and sliced at 10 μm and mounted directly to slides. After blocking with 5% Normal Goat Serum (Vector), rabbit anti-Cas9 primary antibody (Abcam) was applied directly to each section in incubation buffer (1% BSA in PBS) at a 1:100 dilution ratio. The primary antibody was incubated overnight at 4° C. in humidity chambers to prevent drying. Goat anti-rabbit biotinylated secondary (Vector) was applied directly to each section in incubation buffer at a 1:200 dilution ratio. The secondary antibody incubated at room temperature for 1 hour. ABC Elite HRP kit (Vector) was used for detection with methods following the kit's protocol. DAB was used to develop the stain and slides were counterstained with Hemotoxylin. Slides were then dehydrated in ethanol/xylene and permanently mounted with Acrytol. Slides were imaged on LEICA DMIL LED microscope.

### Example 10

### Delivery of Adenoviral Vector Targeting Nrf2 in H1703 (44-25)

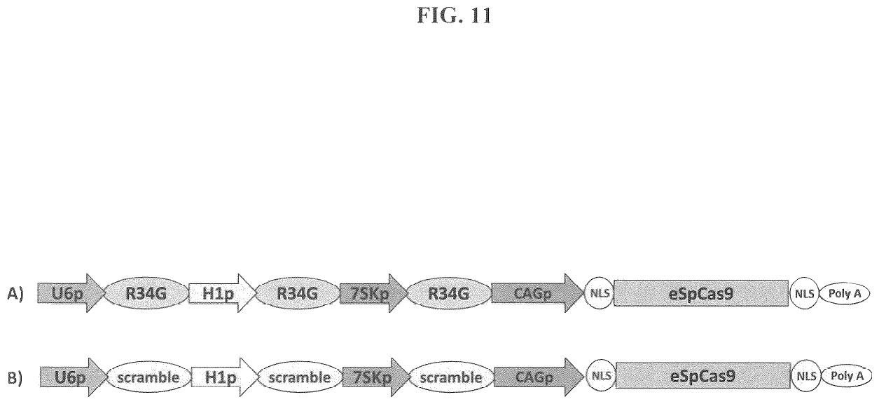

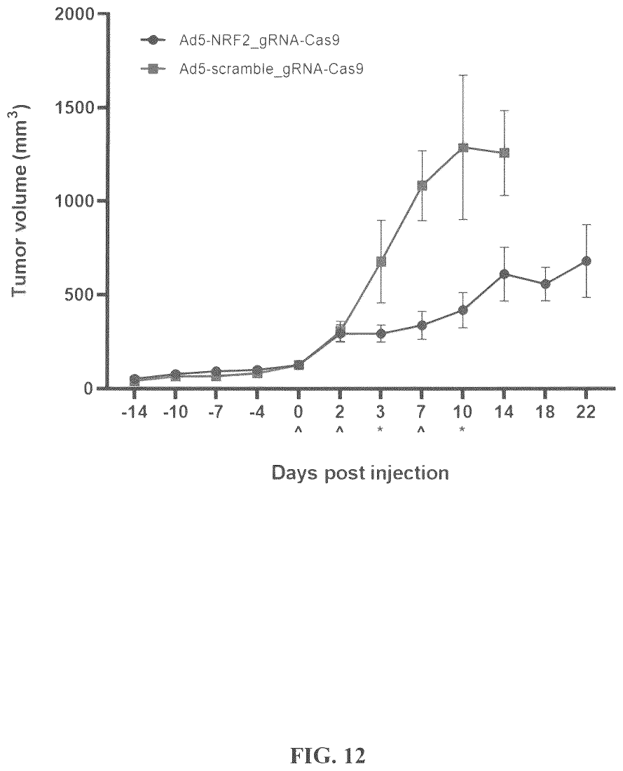

Adenovirus containing CRISPR/Cas9 targeting NRF2 was used to assess intratumoral delivery and efficacy within a xenograft mouse model. CRISPR-engineered human lung squamous cell carcinoma cell line, H1703 44-25, was implanted subcutaneously. Once tumors reached 60-150 mm³ in size, mice were injected intratumorally with 3.6.5e⁹ pfu of A) Ad-U6H17SK-R34G-CAG-eSpCas9 (Vector Biolabs) or B) Ad-U6H17SK-scramble-CAG-eSpCas9 (Vector Biolabs) (as shown in the schematic diagram in FIG. 11) on days 0, 2, and 7 as denoted in FIG. 12 on the x axis by \hat{ } for a total of 1.08e¹⁰ pfu. FIG. 11A shows the schematic of Ad-U6H17SK-R34G-CAG-eSpCas9 with U6, H1 and 7SK promoters driving R34G-targeting sgRNA (GATATAGATCTTGGAGTAAG; SEQ ID NO:25) expression followed by the chicken beta actin (CAG) promoter driving enhanced SpCas9 expression. FIG. 11B shows the schematic of Ad-U6H17SK-scramble-CAG-eSpCas9 with U6, H1 and 7SK promoters driving scramble-targeting sgRNA (GCACTACCAGAGCTAACTCA; SEQ ID NO: 26) expression followed by the chicken beta actin (CAG) promoter driving enhanced SpCas9 expression. Mice were treated with 12.5 mg/kg carboplatin and 5 mg/kg paclitaxel (intravenous injections) on days 3 and 10 as denoted by * on the x axis. Animals were monitored for 22 days post injection. The results show that mice treated with NRF2-targeting adenovirus with combined therapy showed a greater reduction in tumor size at sacrifice. Mice treated with scrambled adenovirus with combined therapy reached endpoint tumor size by day 14. All tumors were collected at sacrifice for further analysis.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of 5×10⁶ cells (human lung squamous cell-derived H1703 clone 44-25) in BD matirgel (volume ratio 1:1). Tumor growth was measured and estimated using a caliper and calculated as volume (mm³)=(length [mm]×(width [mm])²×0.5. When tumors reached ˜60-150 mm³, mice were separated to experimental groups. Adenoviral injections consisted of 3 doses of 3.6e⁹ pfu in a 30 μL of Ad-U6H17SK-R34G-CAG-eSpCas9 or Ad-U6H17SK-scramble-CAG-eSpCas9 purchased from Vector Biolabs and administered every other day. Two doses of 12.5 mg/kg of carboplatin and 5 mg/kg of paclitaxel were administered intravenously in xenografted mice. In some experiments, when tumors reached 1500 mm³ in size, mice were euthanized and tumors were surgically excised and processed for histopathology or genomic DNA.

### Example 11

### Targeting Efficiency of Intratumoral Delivery of Cas9 Using an Adenoviral Vector

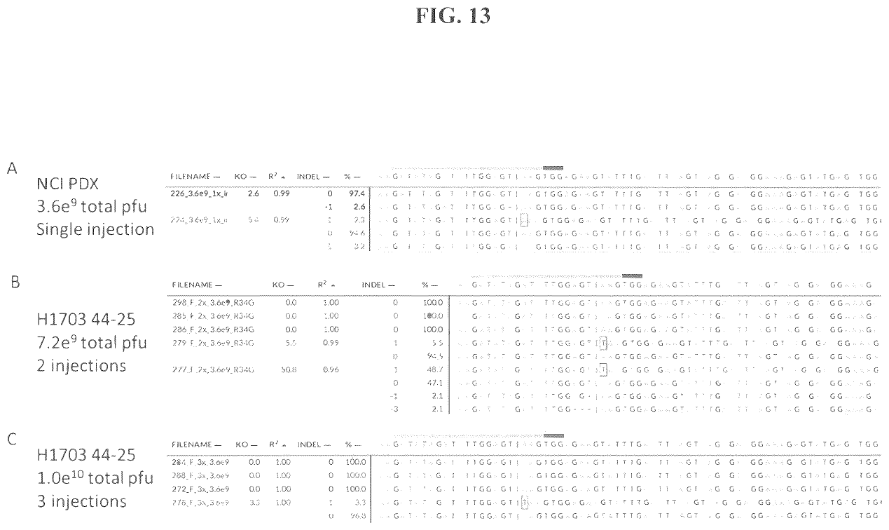

To assess activity and efficiency of NRF2-targeting adenovirus, tumors were excised from experimental mice and genomic DNA was isolated. Once the genomic DNA was isolated, the region of interest-NRF2, was PCR amplified and the PCR amplicon was used for Sanger sequencing. Sanger sequencing from each sample was assessed for indel efficiency at the CRISPR target site, using DECODR software to deconvolute sequence chromatograms. FIG. 13 shows the DECODR analysis output of each sample per experiments. FIG. 13A displays sequencing results from Example 9, which consisted of a single injection of 3.6e⁹ pfu of Ad-U6-R34G-CAG-eSpCas9. The results show two samples with 2.6% and 5.4% CRISPR efficiency. FIG. 13B displays data from experiments not listed but executed similarly to Example 10. The results show 5 samples with 0%, 5.5% and 50.8% CRISPR efficiency. FIG. 13C displays sequencing results from Example 10. The results show 4 samples with 0% and 3.3% CRISPR efficiency.

### Materials and Methods

### Gene Editing Analysis

Cellular genomic DNA was isolated from each clonal cell line using the DNeasy Blood and Tissue Kit (Qiagen). The region surrounding the CRISPR target site was PCR amplified using the Q5 High-Fidelity 2× Master Mix (New England BioLabs) (FWD primer—CACCATCAACAGTGGCATAATGTGAA (SEQ ID NO:27); REV primer—AACTCAGGTTAGGTACTGAACTCATCA (SEQ ID NO:28)). The PCR reaction was purified using the QIAquick PCR Purification Kit (Qiagen) and Big Dye Terminator PCR was performed using Big Dye Terminator v3.1 (Thermofisher). PCR products were purified once more using the Big Dye Xterminator kit (Thermofisher) and then sequenced using the SeqStudio Genetic Analyzer (Applied Biosystems). Sequence analysis was conducted using the software program, DECODR, available on the Decodr website.

### Example 12

### Intratumoral Delivery of LNP/fLuc to H1703 (44-25) SubQ Developed in NCG Mice

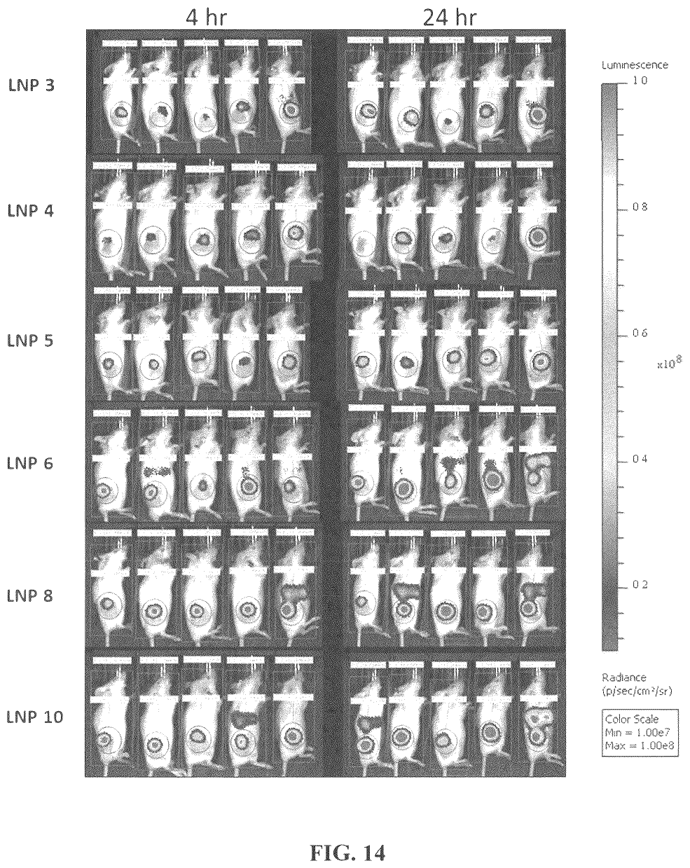

Lipid nanoparticles (LNP) packaged with firefly luciferase mRNA was used to assess intratumoral delivery and expression within a xenograft mouse model. CRISPR-engineered human lung squamous cell carcinoma cell line, H1703 44-25, was implanted subcutaneously. Once tumors reached 60-150 mm³ in size, mice were injected intratumorally with 2 μg of LNP mixture tumor. Bioluminescence imaging was conducted on mice 4 and 24 hours post injection. FIG. 14 displays time course of the bioluminescent signal post intratumoral injection. Signal from each tumor is quantified by establishing a region of interest (ROI) of the tumor. The ROI value listed as total flux photons of each tumor. Four hours post intratumoral injection in mice across all 6 LNPs tested created a strong signal with comparable ROIs. The signal increased at 24 hours post injection.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of 5×10⁶ cells (human lung squamous cell-derived H1703 clone 44-25) in BD matirgel (volume ratio 1:1). Tumor growth was measured and estimated using a caliper and calculated as volume (mm³)=(length [mm]×(width [mm])²×0.5. When tumors reached ˜60-150 mm³, mice were separated to experimental groups. Intratumoral injections consisted of 2 μg of LNP in a total volume of 25 μL purchased from Precision Nanosystems.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 13

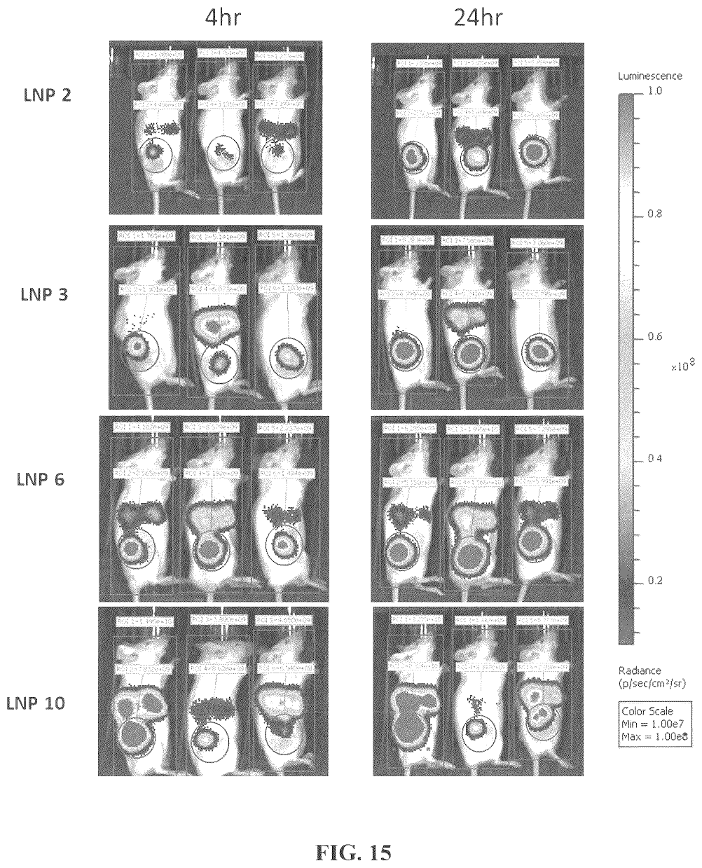

Lipid nanoparticles (LNP) packaged with firefly luciferase mRNA was used to assess intratumoral delivery and expression within a patient-derived xenograft mouse model. Human lung squamous cell carcinoma tumor fragments were implanted subcutaneously. Once tumors reached 60-150 mm³ in size, mice were injected intratumorally with 2 μg of LNP mixture tumor. Bioluminescence imaging was conducted on mice 4 and 24 hours post injection. FIG. 15 displays time course of the bioluminescent signal post intratumoral injection. Signal from each tumor is quantified by establishing a region of interest (ROI) of the tumor. The ROI value listed as total flux photons of each tumor. Four hours post intratumoral injection in mice across all 4 LNPs tested created a strong signal with comparable ROIs. The signal increased at 24 hours post injection.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of PDX fragments derived from human lung squamous cell carcinoma tumor (obtained from Jackson Laboratories, model TM00244). Tumor growth was measured and estimated using a caliper and calculated as volume (mm³)=(length [mm]×(width [mm])²×0.5. When tumors reached ˜60-150 mm³, mice were separated to experimental groups. Intratumoral injections consisted of 2 μg of LNP in a total volume of 25 μL purchased from Precision Nanosystems.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% Isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 14

### Intratumoral Delivery of LNP/fLuc to NCI PDX SubQ Developed in NCG Mice Background

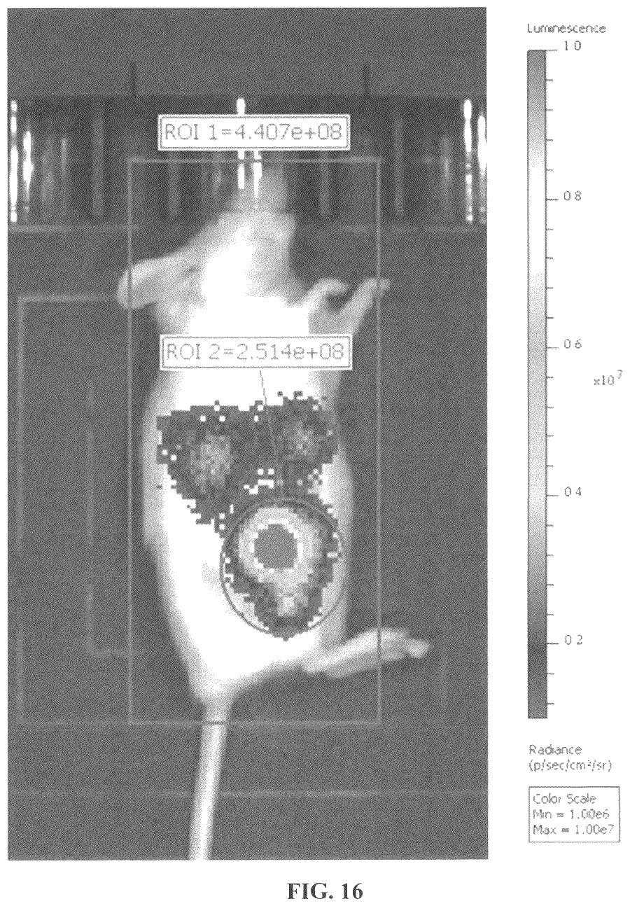

Lipid nanoparticles (LNP) packaged with firefly luciferase mRNA was used to assess intratumoral delivery and expression within a patient-derived xenograft mouse model. Human lung squamous cell carcinoma tumor fragments were implanted subcutaneously. Once tumors reached 60-150 mm³ in size, mice were injected intratumorally with 2 μg of LNP mixture tumor. Bioluminescence imaging was conducted on mice 4 and 24 hours post injection. FIG. 16 displays time course of the bioluminescent signal post intratumoral injection. Signal from each tumor is quantified by establishing a region of interest (ROI) of the tumor. The ROI value listed as total flux photons of each tumor. Twenty-four hours post intratumoral injection in the mouse, the LNP mixture tested created a strong signal with comparable with previous ROIs in Examples 12 and 13.

### Materials and Methods

### Animal Experiments

All experiments with mice conformed to Animal Welfare guidelines and were performed in accordance with protocols approved by University of Delaware's Institutional Animal Care and Use Committee. Tumors were generated in 6-8 week old NCG female mice (Charles River) by subcutaneous implantation of PDX fragments derived from human lung squamous cell carcinoma tumor (obtained from NCI PDMR, specimen ID 073-R). Tumor growth was measured and estimated using a caliper and calculated as volume (mm³)=(length [mm]×(width [mm])²×0.5. When tumors reached ˜60-150 mm³, mice were separated to experimental groups. Intratumoral injections consisted of 2 μg of LNP in a total volume of 25 μL purchased from Precision Nanosystems.

### Bioluminescence Imaging

Bioluminescence imaging was performed with an IVIS Spectrum imaging system (Caliper Life Sciences). Mice were administered intraperitoneally with d-luciferin (Promega) at a dose of 150 mg/kg. Five minutes after receiving d-luciferin, mice were anesthetized in a chamber with 3% isoflurane and placed on the imaging platform while being maintained on 3% isoflurane via a nose cone. Mice were imaged at 15 minutes post administration of d-luciferin using an exposure time of 1 second or longer. Bioluminescence values were quantified by measuring photon flux (photons/second) in the region of interest where bioluminescence signal emanated using the Living IMAGE Software provided by Caliper (Hopkinton, MA).

### Example 15

### AAV Tropism Assessment for AAV5 and AAV6 in a Human Xenograft Mouse Model of H1703 Non-Small Cell Lung Cancer (NSCLC)

Purpose: To determine the in vivo transduction rate of two AAV serotypes, AAV5 and AAV6, expressing firefly luciferase using the H1703 NSCLC human xenograft model in female NCG mice, and assess with bioimaging.

TABLE 7
Treatment Groups

| Gr. | N | Agent | Active dose | Route | Schedule |
|---|---|---|---|---|---|
| 1 | 8 | AAV5-fLUC | 1 × 10¹¹ viral genomes | intra-tumoral | once per |
| | | | (vg)/animal | | day |
| 2 | 8 | AAV6-fLUC | 1 × 10¹¹ viral genomes | intra-tumoral | once per |
| | | | (vg)/animal | | day |

### Procedures

CR female NCG mice were injected with 5×10⁶ H1703 tumor cells in 50% Matrigel subcutaneously in flank. The cell injection volume was 0.1 mL/mouse, and the age of the mice at the start date was 8 to 12 weeks. A pair match was performed when tumors reached an average size of 60-100 mm³ and treatment was begun. An average tumor size of ˜80 mm³ was targeted. Day 1 was defined as the day of dosing of the AAVs. Body weight of the mice was measured daily and tumor size was measured by calipers biweekly until the end of the experiment. Any individual animal with a single observation of >than 30% body weight loss or three consecutive measurements of >25% body weight loss was euthanized. The endpoint of the experiment was a mean tumor weight in the Control Group of 2000 mm³. When the endpoint was reached, all the animals were euthanized.

AAV5-fLUC and AAV6-fLUC containing the firefly luciferase gene under transcriptional control of the chicken actin promoter (CAG) were administered in PBS intratumorally in a volume of 0.05 mL/mouse on Day 1.

Whole body in vivo bioluminescent imaging was performed for a total of 7 imaging timepoints as described below:
All Groups: All Animals—Day 2 (1 day post AAV injection)
All Groups: All Animals—Day 3 (2 days post AAV injection)
All Groups: Animals 2, 3, 5, 6, 7—Day 4 (3 days post AAV injection)
All Groups: Animals 2, 3, 5, 6, 7—Day 6 (4 days post AAV injection)
All Groups: Animals 2, 3, 5, 6, 7—Day 8 (7 days post AAV injection)
All Groups: Animals 2, 3, 5, 6, 7—Day 9 (8 days post AAV injection)
All Groups: Animals 2, 3, 5, 6, 7—Day 10 (9 days post AAV injection)
All Groups: Animals 2, 3, 5, 6, 7—Day 13 (12 days post AAV injection)
All Groups: Animals 2, 3, 5, 6, 7—Day 15 (14 days post AAV injection)
All Groups: Animals 2, 3, 5, 6, 7—Day 17 (16 days post AAV injection)
All Groups: Animals 2, 3, 5, 6, 7—Day 20 (19 days post AAV injection)

The luciferase substrate (D-Luciferin) was administered at 150 mg/kg i.p. at 10 mL/kg (split into 2 injections) based upon recent body weights. Dorsolateral images were taken 10 minutes post substrate injection. Imaging was performed under anesthesia.

For ex vivo bioluminescent imaging, prior to sampling, luciferase substrate (D-Luciferin) was administered at 150 mg/kg i.p. at 10 mL/kg (split into 2 injections) based upon recent body weights. Ex vivo bioluminescent imaging of sampled tissues was performed for a total of 2 imaging timepoints as described below:
All Groups: Animals 1, 4, 8—Day 4 (3 days post AAV injection)
All Groups: Animals 2, 5, 7—Day 21 (20 days post AAV injection)

In order to capture all organs per animal, two images per animal were assumed for a total of 24 images (n=3 animals from each of 2 groups at 2 timepoints, two images per animal).

### RESULTS

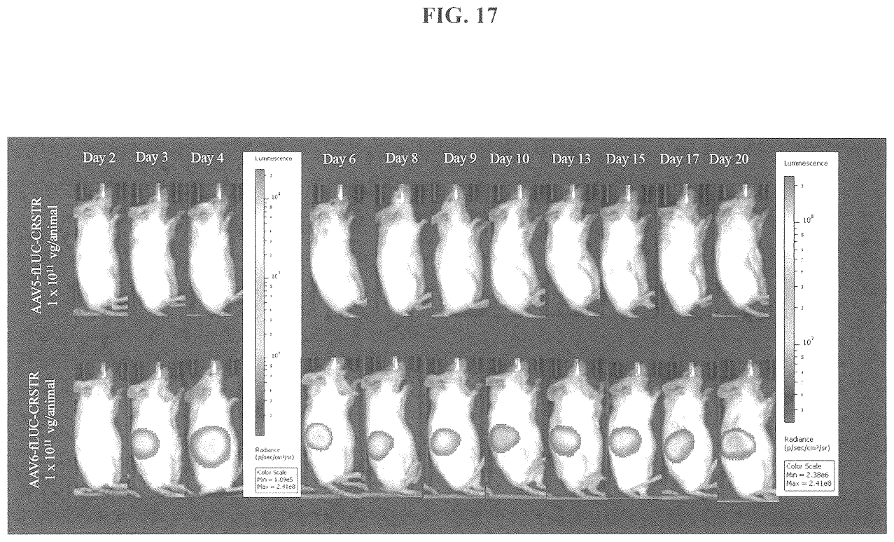

As shown in FIG. 17, mice administered the AAV6-fLUC virus exhibited greater bioluminescence starting at Day 3 relative to mice administered AAV5-fLUC. These results indicate that AAV6 exhibited a greater transduction efficiency for the lung cancer cell line H1703 in vivo relative to AAV5.

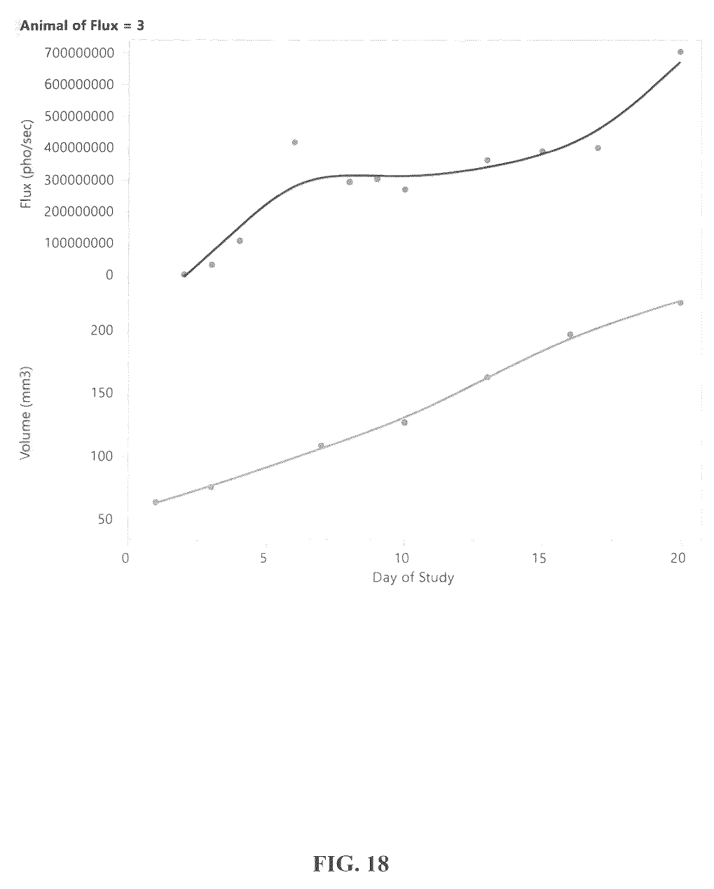

As shown in FIG. 18, both tumor volume and bioluminescence increased over time for a representative mouse implanted with H1703 squamous non-small cell lung carcinoma cells and treated intatumorally with AAV6-fLUC. These results show that bioluminescent signal and tumor volume increased steadily and did not reach a plateau over the duration of the experiment, i.e. 21 days post injection, suggesting that expression of the luciferase gene would continue beyond 21 days post injection.

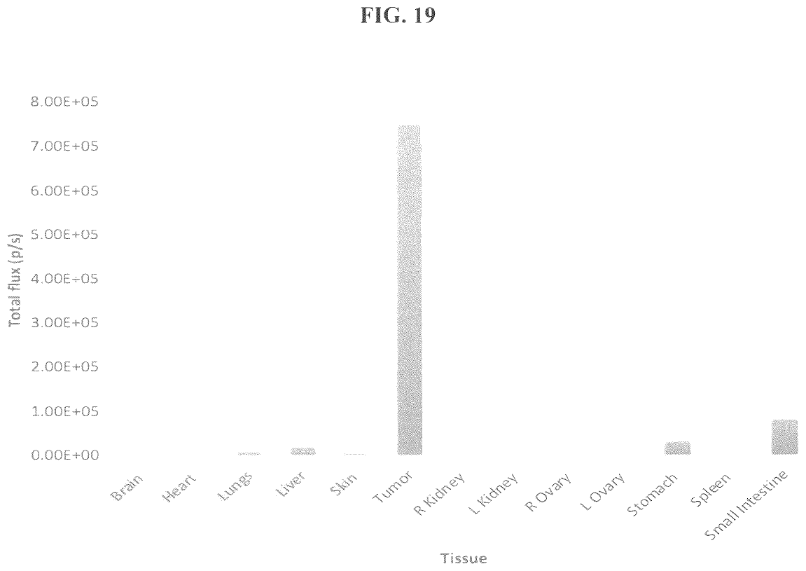

As shown in FIG. 19, bioluminescence was much greater in tumor tissue relative to other tissues on Day 21 in mice treated intatumorally with AAV6-fLUC. These results indicate that the majority of reporter gene expression remains within the tumor in which the AAV is delivered. Qualitatively, these results suggest that there would be less of a possibility of distribution of the AAV to other tissues.

1. A method of treating a solid tumor comprising intratumorally or peritumorally administering to a subject in need thereof a therapeutically effective amount of a CRISPR/Cas system comprising (a) one or more nucleic acid sequences encoding one or more guide RNAs (gRNAs) that are complementary to an NRF2 gene in a target cancer cell and (b) a nucleic acid sequence encoding a CRISPR-associated endonuclease; thereby resulting in at least about a 20% reduction in tumor size as compared to an untreated tumor and/or in at least about a 20% inhibition in tumor growth as compared to an untreated tumor.

2. The method of claim 1, wherein the one or more gRNAs comprise a trans-activated small RNA (tracrRNA) and a CRISPR RNA (crRNA).

3. The method of claim 1, wherein the one or more gRNAs are one or more single guide RNAs.

4.-7. (canceled)

8. The method of claim 1, wherein the CRISPR-associated endonuclease is a class 2 CRISPR-associated endonuclease.

9. The method of claim 8, wherein the class 2 CRISPR-associated endonuclease is Cas9 or Cas12a.

10. The method of claim 1, wherein the CRISPR/Cas system is comprised in a ribonucleoprotein (RNP) or lipid nanoparticle (LNP) complex.

11. The method of claim 1, wherein the CRISPR/Cas system is comprised in one or more vectors that drive expression of one or more elements of the CRISPR system.

12. The method of claim 11, wherein the one or more vectors is a viral vector, a liposome, or a lipid-containing complex.

13. The method of claim 12, wherein the viral vector is an adenovirus, an adenovirus-associated virus (AAV), a helper-dependent adenovirus, a retrovirus, or a hemagglutinating virus of Japan-liposome (HVJ) complex.

14. The method of claim 1, wherein the solid tumor is an adenoid cystic carcinoma tumor, a biliary tract cancer tumor, a bladder cancer tumor, a bone cancer tumor, a breast cancer tumor, a cervical cancer tumor, a cholangiocarcinoma tumor, a colon cancer tumor, an endometrial cancer tumor, an esophageal cancer tumor, a gallbladder cancer tumor, a gastric cancer tumor, a head and neck cancer tumor, a hepatocellular cancer tumor, a kidney cancer tumor, a lip cancer tumor, a liver cancer tumor, a melanoma tumor, a mesothelioma tumor, a non-small cell lung cancer tumor, a nonmelanoma skin cancer tumor, an oral cancer tumor, an ovarian cancer tumor, a pancreatic cancer tumor, a prostate cancer tumor, a rectal cancer tumor, a renal cancer tumor, a sarcoma tumor, a small cell lung cancer tumor, a spleen cancer tumor, a thyroid cancer tumor, a urothelial cancer tumor, or a uterine cancer tumor.

15. A method of reducing expression of NRF2 in a cancer cell of a solid tumor comprising intratumorally or peritumorally introducing into the cancer cell (a) one or more nucleic acid sequences encoding one or more guide RNAs (gRNAs) that are complementary to an NRF2 gene in the NRF2 gene and (b) a nucleic acid sequence encoding a CRISPR-associated endonuclease, whereby the one or more gRNAs hybridize to the NRF2 gene and the CRISPR-associated endonuclease cleaves the NRF2 gene; thereby resulting in at least about a 20% reduction in tumor size as compared to an untreated tumor and/or in at least about a 20% inhibition in tumor growth as compared to an untreated tumor.

16. The method of claim 15, wherein the one or more gRNAs comprise a trans-activated small RNA (tracrRNA) and a CRISPR RNA (crRNA).

17. The method of claim 15, wherein the one or more gRNAs are one or more single guide RNAs.

18. The method of claim 15, wherein the CRISPR-associated endonuclease is a class 2 CRISPR-associated endonuclease.

19. The method of claim 18, wherein the class 2 CRISPR-associated endonuclease is Cas9 or Cas12a.

20.-22. (canceled)

23. The method of claim 15, wherein the one or more nucleic acid sequences of (a) and the nucleic acid sequence of (b) is comprised in an RNP or LNP complex.

24. The method of claim 15, wherein the CRISPR/Cas system is comprised in one or more vectors that drive expression of one or more elements of the CRISPR system.

25. The method of claim 24, wherein the one or more vectors is a viral vector, a liposome, or a lipid-containing complex.

26. The method of claim 25, wherein the viral vector is an adenovirus, an AAV, a helper-dependent adenovirus, a retrovirus, or a hemagglutinating virus of HVJ complex.

27. The method of claim 15, wherein the solid tumor is an adenoid cystic carcinoma tumor, a biliary tract cancer tumor, a bladder cancer tumor, a bone cancer tumor, a breast cancer tumor, a cervical cancer tumor, a cholangiocarcinoma tumor, a colon cancer tumor, an endometrial cancer tumor, an esophageal cancer tumor, a gallbladder cancer tumor, a gastric cancer tumor, a head and neck cancer tumor, a hepatocellular cancer tumor, a kidney cancer tumor, a lip cancer tumor, a liver cancer tumor, a melanoma tumor, a mesothelioma tumor, a non-small cell lung cancer tumor, a nonmelanoma skin cancer tumor, an oral cancer tumor, an ovarian cancer tumor, a pancreatic cancer tumor, a prostate cancer tumor, a rectal cancer tumor, a renal cancer tumor, a sarcoma tumor, a small cell lung cancer tumor, a spleen cancer tumor, a thyroid cancer tumor, a urothelial cancer tumor, or a uterine cancer tumor.

28.-31. (canceled)

32. The method of claim 1, further comprising administering one or more chemotherapeutic agents to the subject.

33. The method of claim 32, wherein intratumoral or peritumoral delivery of the CRISPR/Cas system reduces the amount of one or more chemotherapeutic agents administered to the subject as compared to a subject that does not receive administration of the CRISPR/Cas system.
